(19) United States
(12) Patent Application Publication
(10) Pub. No.: US 2010/0197914 A1
(43) Pub. Date: Aug. 5, 2010

(54) Purine Derivatives as Adenosine Al Receptor Ligands

(76) Inventor: Robin Alec Fairhurst, West Sussex (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/679,663

(22) PCT Filed: Oct. 15, 2008
(86) PCT No.: PCT/EP08/63871

§ 371 Date: Mar. 23, 2010

(30) Foreign Application Priority Data
Oct. 17, 2007 (EP) 07118719.9

(51) Int. Cl. C07D 473/34; C07D 473/40; C07D 473/16

(52) U.S. Cl. 544/277

(57) ABSTRACT

Compounds of formula (I), their preparation and use as pharmaceuticals (I), wherein X, Y, and Z are as defined herein.

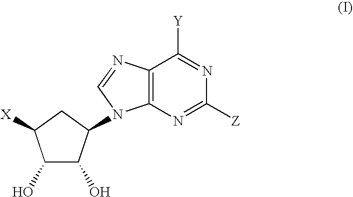

This invention relates to organic compounds, their preparation and use as pharmaceuticals. In particular, this invention relates to adenosine receptor ligand compounds, and their use as adenosine A₁ receptor ligands and, in particular as adenosine A₁ receptor agonists, of both high and low intrinsic efficacy, for the treatment of diseases such as sleep disorders, hypertension, myocardial ischemia, epilepsy, chronic inflammatory pain, irritable bowel syndrome, nausea, obesity and/or type 2 diabetes, preferably when administered by the oral route.

In one aspect, the present invention provides compounds of formula (I)

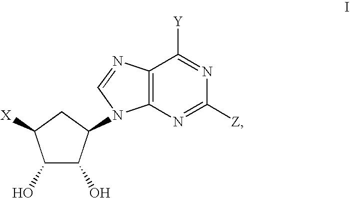

or stereoisomers thereof, in free or pharmaceutically acceptable salt form, wherein
X denotes —NHC(O)R¹, —NHC(O)OR², —N-bonded HET¹ or NHC(O)—NR³R⁴,
   wherein R¹ and R² are independently selected from the group including H, C₁-C₈ alkyl, C₂-C₈ alkenyl, C₂-C₈ alkynyl, C₁-C₈ alkoxy, and C₃-C₈ cycloalkyl, and wherein said alkyl, alkenyl, alkynyl, alkoxy or cycloalkyl groups of R¹ and R² may optionally be substituted by one or more substituents independently selected from the group including NH₂, OH, and OR⁵, and wherein R⁵ is a C₁-C₃ alkyl group;
   wherein R³ and R⁴ are independently selected from the group including H, and C₁-C₄ alkyl;
   wherein said HET¹ group is an N-bonded 4- to 6-membered heterocyclic group containing from 1 to 4 nitrogen atoms and may optionally be benzo-fused, and wherein HET¹ may optionally be substituted by one or more groups independently selected from the group including H, C₁-C₃ alkyl, C₁-C₃ alkoxy, and —C(O), and wherein said alkyl and alkoxy groups may optionally be further substituted by —NH₂ or —OH;
Y denotes —NH₂, —NHR⁶, —N(R⁶)₂, —NHR⁶(aryl), —NHR⁷(HET²), —NHR⁸, —NHC(O)R⁸, or —NH(HET³),
   wherein R⁶ is C₁-C₈ alkyl, C₂-C₈ alkenyl, C₂-C₈ alkynyl, C₁-C₈ alkoxy, or C₃-C₈ cycloalkyl group, and wherein said cycloalkyl group may be saturated or unsaturated, fused or bridged, and wherein said alkyl, alkenyl, alkynyl, alkoxy, or cycloalkyl groups of R⁶ may be optionally substituted by one or more groups independently selected from the group including OH, halogen, —C₁-C₆alkoxy, —C₁-C₆alkyl, —O-aryl, and an —S—(S-HET) heterocyclic group, and wherein —(S-HET) is a C-bonded 5- to 8-membered ring system having one or two heteroatoms selected from O, N and S, and wherein —(S-HET) may be optionally substituted by one or more groups independently selected from halogen, and C₁-C₈ alkyl;
   wherein the heterocyclic group of —NHR⁷(HET²) is a C-bonded 5- or 6-membered heterocyclic group containing one or two heteroatoms selected from O, N or S, and wherein HET² may optionally be substituted by one or more substitutents independently selected from the group including halogen, C₁-C₆alkyl, and —C(O)C₁-C₆alkyl;
   and wherein the R^{7a} group is a C₁-C₈ alkyl group which may be optionally substituted by a C₁-C₃ alkyl group;
   wherein the heterocyclic group of —NH(HET³) is a C or N-bonded 5- or 6-membered heterocyclic group containing one or two heteroatoms selected from O, N and S, and wherein HET³ may optionally be substituted by one or more substituents independently selected from the group including halogen, —C₁-C₆alkyl, —C(O)O(C₁-C₆alkyl), —S-aryl, and a —C-bonded 5- or 6-membered heterocyclic group containing one or two N heteroatoms (C-HET) wherein C-HET¹ is optionally substituted by one or more CF₃ substitutents;
   wherein R⁸ is an aryl group,
   wherein the aryl group of —NHR⁸ is either mono-substituted with —OH, halogen or —C₁-C₆alkyl, or di-substituted with two groups independently selected from the group including —OH, halogen, —C₁-C₆alkyl, —N(—C₁-C₆alkyl)₂, and —NH(HET⁴); or is tri-substituted with three groups independently selected from the group including —OH, halogen, and —C₁-C₆alkyl;
   wherein said HET⁴ group is a C-bonded 5- or 6-membered heterocyclic group containing one or two heteroatoms selected from O, N and S, and wherein HET⁴ may optionally be substituted by one or more groups independently selected from the group including H, halogen, —C₁-C₆alkyl, aryl, heteroaryl, —C₁-C₆alkoxy, —O-aryl, —N(C₁-C₆alkyl), —N(aryl), and —N(heteroaryl);
   wherein the aryl group of —NHC(O)R⁸ may be optionally substituted by one or more aryl groups;
Z denotes H, halogen, HET⁵, or —N═N—NHC(O)—NH-aryl, wherein said HET⁵ group is a 5- or 6-membered ring containing from one to four N heteroatoms, and wherein HET⁵ may optionally be substituted by one or more groups independently selected from the group including —C₁-C₆alkyl-C(O)R^{x}, —C(O)R^{x}, —C(O)NHR^{y}, —NHC(O)R^{X}, a C-bonded 5- or 6-membered ring containing from one or two N heteroatoms (HET⁵), and aryl;
   wherein R^{x} is selected from the group including H, OH, C₁-C₆alkyl, —O(C₁-C₆alkyl), and aryl, wherein said aryl group may be optionally substituted by halogen or C₁-C₃alkyl; and
   wherein R^{y} is selected from the group including H, C₁-C₆alkyl, aryl, and C₁-C₆alkyl(aryl), wherein said aryl groups may be optionally substituted by one or more CF₃ groups.
   According to an embodiment of the present invention, there is provided compounds of formula (I) as defined hereinbefore with the provisos that:

(a) when X is —NHC(O)Me and Y is -3-iodobenzylamino then Z is not Cl or H; and

(b) when X is —N-bonded [1,2,3]triazol-2-yl and Y is -3-iodobenzylamino then Z is not Cl.

In another embodiment, the present invention provides compounds of formula (I)

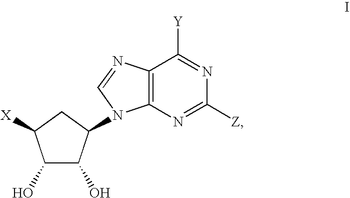

or stereoisomers, in free or pharmaceutically acceptable salt form,
wherein
X is —NHC(O)R¹, —NHC(O)OR², N-bonded(HET¹), or —NHC(O)—NR³R⁴;
   wherein R¹ and R² are independently selected from the group including C₁-C₄ alkyl, C₁-C₃ alkoxy, and C₃-C₄ cycloalkyl, and wherein said alkyl, alkoxy or cycloalkyl groups may optionally be substituted by one or more substituents independently selected from NH₂, and OH;
   wherein R³ and R⁴ are independently selected from H, and methyl;
   wherein HET¹ is an, optionally benzo-fused, N-bonded 5- to 6-membered heterocyclic group containing from 1 to 4 N heteroatoms, and wherein HET¹ may optionally be substituted by one or more groups independently selected from the group including H, methyl, ethyl, i-propyl, n-propyl, —CH₂OH, —OCH₃, —CH₂CH₂OH, —CH₂NH₂, —CH(CH₃)OH, and —C(O); and wherein
Y is —NH₂, —NHR⁶, —N(R⁶)₂, —NHR⁷(HET²), —NHR⁸, —NHC(O)R⁸, or —NH(HET³), wherein R⁶ is C₁-C₄ alkyl, or C₃-C₈ cycloalkyl wherein said cycloalkyl group may be saturated, fused or bridged; and
   wherein, when Y is —NHR⁶, R⁶ is selected from the group including Me, Et, iPr, nPr, iBu, nBu, tBu, and C₃-C₈ cycloalkyl, or R⁶ is a C₁ to C₄ alkyl group substituted by —S—(S-HET) or —O-aryl; and
   wherein, when Y is —N(R⁶)₂, R⁶ is C₃-C₅ cycloalkyl, and
   wherein said alkyl, or cycloalkyl groups of NHR⁶ and N(R⁶)₂ may be optionally substituted by one or more groups independently selected from the group including halogen, —C₁-C₃alkoxy, —C₁-C₃alkyl, —O-aryl, and —S—(S-HET), and
   wherein, when Y is —NHR⁷(HET²), R⁷ is C₁-C₄ alkyl and HET² is a C-bonded 5-membered heterocyclic group containing one heteroatom selected from O, S or N, and wherein HET² may optionally be substituted by one or more substitutents independently selected from the group including Cl, F, Me, and Et, and wherein the alkyl group of —NHR⁷(HET²) is optionally substituted by a C₁-C₃ alkyl group; and
   wherein the 5- to 6-membered heterocyclic group of —NH(HET³) is C- or N-bonded and contains one or two heteroatoms selected from O, and N, and, may optionally be substituted by one or more substituents independently selected from the group including Cl, F, —C₁-C₃alkyl, —C(O)O(C₁-C₃alkyl), —S-phenyl, and —C-HET¹ wherein —C-HET¹ is a C-bonded 6-membered heterocyclic group containing one N heteroatom and wherein —C-HET¹ is optionally substituted by one or more —CF₃ substitutents,
   preferably -HET³ is a C-bonded 5- or 6-membered heterocyclic group including an O heteroatom
   wherein R⁸ is a phenyl group; and
   wherein the phenyl group of —NHR⁸ is either: mono-substituted with —OH, F, Cl, —C₁-C₃alkyl, or —CH₂C(O)NH-phenyl-C(O)NH—CH₂NH₂; or is di-substituted with two groups independently selected from the group including —OH, F, Cl, and —C₁-C₃alkyl; or is tri-substituted with three groups independently selected from the group including —CH₃, F, and, Cl, and
   wherein the phenyl group of —NHC(O)R⁸ may be optionally substituted by one or more aryl groups; and wherein
   Z is H, Cl, F or HET⁵ wherein HET⁵ is an N-bonded 5-membered heterocyclic group containing one or two N heteroatoms, and wherein HET⁵ is optionally substituted by one or more groups independently selected from —C(O)R^{x}, —C(O)NHR^{y} and a —C-bonded 6-membered heterocyclic group containing one or two N heteroatoms (HET⁵); and
   wherein R^{x} is —OMe, —OEt, OH, or phenyl, and
   wherein R^{y} is H, Me, Et, phenyl substituted by CF₃, or C₁-C₃ alkylphenyl substituted by CO₂H, Me or CF₃.
      According to a further embodiment, the present invention provides compounds of formula (I) wherein
X is suitably —NHC(O)R¹, or an N-bonded (HET¹) group,
   wherein R¹ is selected from the group including Me, Et, -EtOH, and -MeOH, and wherein HET¹ is an N-bonded tetrazolyl, pyrazolyl, triazolyl, indazolyl (benzopyrazolyl), 2,4-di-keto-imidazolyl, or 2-keto-pyridinyl group, and
   wherein HET¹ is suitably an N-bonded tetrazolyl, pyrazolyl, or triazolyl group, and wherein said R¹ or HET¹ groups may be mono-substituted by a substitutent independently selected from the group including OH, Me, Et, MeOH, and EtOH; and wherein
Y is suitably —NHR⁶, —NHR⁷(HET²), —NHR⁸, —NHC(O)R⁸, or —NH(HET³),
   wherein R⁶ is ethyl, n-propyl, i-propyl, n-butyl, i-butyl, t-butyl, cyclopentyl, cyclohexyl, or norbornane (bicyclic[2.2.1]heptane), and
   suitably R⁶ is ethyl, cyclopentyl, cyclohexyl, or norbornane (bicyclic[2.2.1]heptane)
   wherein said R⁶ alkyl groups of —NHR⁶ may independently be optionally substituted by one or more groups independently selected from the group including C₁-C₃ alkyl, —S—(S-HET), —O-phenyl, and NH(C₅-C₇)cycloalkyl,
   Optionally, the alkyl group of —NHR⁶ is ethyl substituted by one or more groups independently selected from the group including C₁-C₂ alkyl, S—(S-HET), —O-phenyl, and NH(C₅-C₇)cycloalkyl,
   wherein said R⁶ cycloalkyl groups of —NHR⁶ may independently be optionally substituted by one or more groups independently selected from the group including —OH, —OCH₃, —O-aryl, and —S-benzothiazole (benzthiazole); and
   wherein HET² is thiophene, optionally substituted by one or more substitutents independently selected from the group including Cl, and F, and wherein
   wherein HET³ is tetrahydropyran, tetrahydrofuran or pyrrolidine, each of which may be optionally substituted by one or more substituents independently selected from the group including Cl, F, and a pyridinyl group, wherein said pyridinyl group may be a pyridin-2-yl group and is optionally substituted by one or more substitutents independently selected from the group including CF₃, Cl and F, and
   wherein R⁸ is a phenyl group,
   wherein the phenyl group of —NHR⁸ is either mono-substituted with —OH, F, Cl, or —C₁-C₃alkyl, or is di-substituted with two groups independently selected from the group including —OH, F, and Cl; and
   wherein the phenyl group of —NHC(O)R⁸ may be optionally substituted by a phenyl group; and wherein
Z is suitably H, Cl or a 1H-pyrazole group (HET⁵), wherein said HET⁵ group may be optionally substituted by —C(O)NHR_{y}, or HET⁵, wherein R^{y} is H, Me or —CH₂-phenyl-CO₂H, and
   wherein HET⁶ is a C-bonded pyridin-2-yl group.

Suitable X groups for use according to the present invention are selected from the group including propionamide, 2-hydroxy-acetamide, 5-ethyltetrazole, 4-hydrozymethylpyrazole, acetamide, and 4-methyl-[1,2,3]triazole.

Optionally, X groups suitable for use according to the present invention may be selected from the group including propionamide, 2-hydroxy-acetamide, and 4-hydrozymethylpyrazole.

Furthermore, X groups suitable for use according to the present invention may be selected from the group including propionamide and 2-hydroxy-acetamide.

Thus, according to a further aspect the present invention provides compounds of formula (I) wherein X is as defined anywhere hereinbefore.

Suitable Y groups for use according to the present invention are selected from the group including cyclopentylamino, tetrahydropyran-4-yamino, (S)-2-methoxy-cyclopentylamino, 3-fluoro-4-hydroxy-phenylamino, (S)-norbornaneamino [(S)-(bicyclo[2.2.1]heptaneamino)], (1S,2S)-2-methoxycyclopentylamino, (1S,2S) hydroxycyclopentylamino, tetrahydro-2H-pyran-4-amino, 3-fluoro-4-hydroxy-phenylamino, (R)-(tetrahydro-furan-3-yl)amino, (R)-1-(3-chloro-thiophen-2-ylmethyl)-propylamino, (S)-1-(5-trifluoromethyl-pyridin-2-yl)-pyrrolidin-3-yl-amino, 4-({4-[(2-amino-ethylcarbamoyl)-methyl]-phenylcarbamoyl)-methyl)-phenylaminoyl}, cyclohexylamino, (R)-1-(4-chloro-thiophene-3-yl)amino, (R)-2-(benzothiazole-2-ylsulfanyl)-1-methyl-ethylamino, biphenyl-4-carboxylicacid-amino, (R)-1-methyl-2-phenoxy-ethylamino, and 4-phenylsulfonyl-piperidin-1-ylamino.

Optionally, Y groups suitable for use according to the present invention may be selected from the group including cyclopentylamino, (S)-2-methoxy-cyclopentylamino, (S)-(bicyclo[2.2.1]heptaneamino), (1S,2S)-2-methoxycyclopentylamino, (1S,2S) hydroxycyclopentylamino, (R)-(tetrahydro-furan-3-yl)amino, and (R)-1-(3-chloro-thiophen-2-ylmethyl)-propylamino.

Furthermore, Y groups suitable for use according to the present invention may be selected from the group including cyclopentylamino.

Thus, according to a further embodiment, the present invention provides compounds of formula (I) wherein Y is as defined anywhere hereinbefore.

Suitable Z groups for use according to the present invention are selected from the group including H, Cl, 1H-pyrazole-4-carboxylic acid amide, 1H-pyrazole-4-carboxylic acid, (1H-pyrazole-4-carbonyl-amino)-methyl-benzoic acid, pyrazol-1-yl, 4-pyridin-2-yl-pyrazol-1-yl, 1H-pyrazole-4-carboxylic acid methyl amide, and [(phenylamino)carboyl]-1-trizenyl.

Optionally, Z groups suitable for use according to the present invention may be selected from the group including H, Cl, 1H-pyrazole-4-carboxylic acid amide, 1H-pyrazole-4-carboxylic acid, and 1H-pyrazole-4-carboxylic acid methyl amide.

Furthermore, Z groups suitable for use according to the present invention may be selected from the group including H, Cl, and 1H-pyrazole-4-carboxylic acid amide.

Thus, according to a further aspect the present invention provides compounds of formula (I) wherein Z is as defined anywhere hereinbefore.

In a further embodiment, the present invention provides compounds of formula (IA)

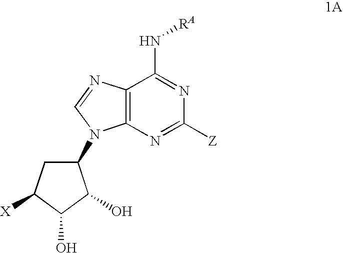

Wherein X and Z are as defined hereinbefore and wherein Y is NH(R^{A}) wherein R^{A} is R⁶, R⁶(aryl),
R⁷(HET²), or HET³, and wherein R⁶, R⁷HET² and HET³ are as defined hereinbefore.

In another embodiment, the present invention provides compounds of formula (I) or (IA) wherein
X is selected from the group including propionamide, 2-hydroxy-acetamide, 5-ethyltetrazole, 4-hydrozymethylpyrazole, acetamide, and 4-mathyl-[1,2,3]triazole; and wherein
Y is selected from the group including cyclopentylamino, tetrahydropyran-4-yamino, (S)-2-methoxy-cyclopentylamino, 3-fluoro-4-hydroxy-phenylamino, (S)-norbornaneamino [(S)-(bicyclo[2.2.1]heptaneamino)], (1S,2S)-2-methoxycyclopentylamino, (1S,2S) hydroxycyclopentylamino, tetrahydro-2H-pyran-4-amino, 3-fluoro-4-hydroxy-phenylamino, (R)-(tetrahydro-furan-3-yl)amino, (R)-1-(3-chloro-thiophen-2-ylmethyl)-propylamino, (S)-1-(5-trifluoromethyl-pyridin-2-yl)-pyrrolidin-3-yl-amino, 4-({4-[(2-amino-ethylcarbamoyl)-methyl]-phenylcarbamoyl}-methyl)-phenylamino, cyclohexylamino, (R)-1-(4-chloro-thiophene-3-yl)amino, (R)-2-(benzothiazole-2-ylsulfanyl)-1-methyl-ethylamino, biphenyl-4-carboxylicacid-amino, (R)-1-methyl-2-phenoxy-ethylamino, and 4-phenylsulfonyl-piperidin-1-ylamino; and wherein
Z is selected from the group including H, Cl, 1H-pyrazole-4-carboxylic acid amide, 1H-pyrazole-4-carboxylic acid, (1H-pyrazole-4-carbonyl-amino)-methyl-benzoic acid, pyrazol-1-yl, 4-pyridin-2-yl-pyrazol-1-yl, 1H-pyrazole-4-carboxylic acid methyl amide, and [(phenylamino)carboyl]-1-trizenyl.

In another embodiment, the present invention provides compounds of formula (I) or (IA) wherein
X is selected from the group including propionamide, 2-hydroxy-acetamide, and 4-hydrozymethylpyrazole; and wherein
Y is selected from the group including cyclopentylamino, (5)-2-methoxy-cyclopentylamino, (5)-(bicyclo[2.2.1]heptaneamino), (1S,2S)-2-methoxycyclopentylamino, (1S,2S) hydroxycyclopentylamino, (R)-(tetrahydro-furan-3-yl)amino, and (R)-1-(3-chloro-thiophen-2-ylmethyl)-propylamino; and wherein
Z is selected from the group including H, Cl, 1H-pyrazole-4-carboxylic acid amide, 1H-pyrazole-4-carboxylic acid, and 1H-pyrazole-4-carboxylic acid methyl amide.

In another embodiment, the present invention provides compounds of formula (I) or (IA) wherein
X is selected from the group including propionamide and 2-hydroxy-acetamide, and wherein Y is selected from the group including cyclopentylamino; and wherein Z is selected from the group including H, Cl, and 1H-pyrazole-4-carboxylic acid amide.

In another embodiment, the present invention provides compounds of formula (I) independently selected from:
N-[(1S,2R,3S,4R)-4-(6-Cyclopentylamino-purin-9-yl)-2,3-dihydroxy-cyclopentyl]-propionamide;
N-[(1S,2R,3S,4R)-4-(2-Chloro-6-cyclopentylamino-purin-9-yl)-2,3-dihydroxy-cyclopentyl]-propionamide;
N-[(1S,2R,3S,4R)-4-(6-Cyclopentylamino-purin-9-yl)-2,3-dihydroxy-cyclopentyl]-2-hydroxy-acetamide;
N-[(1S,2R,3S,4R)-4-(2-Chloro-6-cyclopentylamino-purin-9-yl)-2,3-dihydroxy-cyclopentyl]-2-hydroxy-acetamide;
1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride;
N-{(1S,2R,3S,4R)-2,3-Dihydroxy-4-[6-(tetrahydro-pyran-4-ylamino)-purin-9-yl]-cyclopentyl}-propionamide;
N-{(1S,2R,3S,4R)-2,3-Dihydroxy-4-[6-((S)-2-methoxy-cyclopentylamino)-purin-9-yl]-cyclopentyl}-propionamide;
N-{(1S,2R,3S,4R)-4-[6-(3-Fluoro-4-hydroxy-phenylamino)-purin-9-yl]-2,3-dihydroxy-cyclopentyl}-propionamide;
N-{(1S,2R,3S,4R)-2,3-Dihydroxy-4-[6-((1R,2S,4S)-bicyclo[2.2.1]heptan-2-amino)-purin-9-yl]-cyclopentyl}-propionamide,
(1R,2S,3R,5S)-3-[2-Chloro-6-[(1S,2S)-2-methoxycyclopentylamino]-purin-9-yl]-5-(5-ethyl-tetrazol-2-yl)-cyclopentane-1,2-diol;
(1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol;
1-[6-{tetrahydro-2H-pyran-4-amino}-9-((1R,2S,3R,4S)-2,3-dihydroxy-4-propionylamino-cyclopentyl)-9H-purin-2-yl]-1H-pyrazole-4-carboxylic acid tetrahydro-2H-pyran-4-amine
4-[({1-[6-[(1S,2S)-2-methoxycyclopentylamino]-9-((1R,2S,3R,4S)-2,3-dihydroxy-4-propionylamino-cyclopentyl)-9H-purin-2-yl]-1H-pyrazole-4-carbonyl}-amino)-methyl]-benzoic acid;
1-[9-[(1R,2S,3R,4S)-2,3-Dihydroxy-4-(2-hydroxy-acetylamino)-cyclopentyl]-6-(3-fluoro-4-hydroxy-phenylamino)-9H-purin-2-yl]-1H-pyrazole-4-carboxylic acid amide;
1-{6-[(1S,2S)-2-methoxycyclopentylamino]-9-[(1R,2S,3R,4S)-2,3-dihydroxy-4-(5-methyl-tetrazol-2-yl)cyclopentyl]9Hpurin-2-yl}-1H-pyrazole-4-carboxylic acid amide;
N-[(1S,2R,3S,4R)-4-(6-(1R,2S,4S)-bicyclo[2.2.1]heptan-2-amino-2-pyrazol-1-yl-purin-9-yl)-2,3-dihydroxy-cyclopentyl]-2-hydroxy-acetamide;
(1R,2S,3R,5S)-3-[6-((1S,2S)-2-Hydroxy-cyclopentylamino)-2-(4-pyridin-2-yl-pyrazol-1-yl)-purin-9-yl]-5-(4-hydroxymethyl-pyrazol-1-yl)-cyclopentane-1,2-diol;
1-{9-[(1R,2S,3R,4S)-2,3-Dihydroxy-4-(2-hydroxy-acetylamino)-cyclopentyl]-6-[(R)-(tetrahydro-furan-3-yl)amino]-9H-purin-2-yl}-1H-pyrazole-4-carboxylic acid methylamide;
1-[9-[(1R,2S,3R,4S)-2,3-Dihydroxy-4-(2-hydroxy-acetylamino)-cyclopentyl]-6-((1S,2S)-2-methoxy-cyclopentylamino)-9H-purin-2-yl]-1H-pyrazole-4-carboxylic acid amide;
N-((1S,2R,3S,4R)-4-{6-[(R)-1-(3-Chloro-thiophen-2-ylmethyl)-propylamino]-purin-9-yl}-2,3-dihydroxy-cyclopentyl)-acetamide;
(1S,2R,3S,5R)-3-(4-Methyl-[1,2,3]triazol-2-yl)-5-{6-[(S)-1-(5-trifluoromethyl-pyridin-2-yl)-pyrrolidin-3-ylamino]-purin-9-yl}-cyclopentane-1,2-diol;
N-((1S,2R,3S,4R)-4-{6-[4-({4-[(2-Amino-ethylcarbamoyl)-methyl]-phenylcarbamoyl}-methyl)-phenylamino]-purin-9-yl}-2,3-dihydroxy-cyclopentyl)-propionamide;
N-[(1S,2R,3S,4R)-4-(6-((1R,2S,4S)-bicyclo[2.2.1]heptan-2-amino)-purin-9-yl)-2,3-dihydroxy-cyclo pentyl]-propionamide;
N-[(1S,2R,3S,4R)-4-(6-Cyclohexylamino-purin-9-yl)-2,3-dihydroxy-cyclopentyl]-propionamide;
N-((1S,2R,3S,4R)-4-{6-[(R)-1-(4-Chloro-thiophen-3-ylmethyl)-propylamino)-purin-9-yl]-2,3-dihydroxy-cyclopentyl}-propionamide;
N-((1S,2R,3S,4R)-2,3-Dihydroxy-4-{6-[(R)-(tetrahydro-furan-3-yl)amino]-purin-9-yl}-cyclopentyl)-propionamide;
N-((1S,2R,3S,4R)-4-{6-[(R)-2-(Benzothiazol-2-ylsulfanyl)-1-methyl-ethylamino]-2-chloro-purin-9-yl}-2,3-dihydroxy-cyclopentyl)-propionamide;
N-{(1S,2R,3S,4R)-2,3-Dihydroxy-4-[6-((1S,2S)-2-hydroxy-cyclopentylamino)-purin-9-yl]-cyclopentyl}-propionamide;
Biphenyl-4-carboxylic acid [9-((1R,2S,3R,4S)-2,3-dihydroxy-4-propionylamino-cyclopentyl)-9H-purin-6-yl]-amide;
N-{(1S,2R,3S,4R)-4-[2-Chloro-6-((R)-1-methyl-2-phenoxy-ethylamino)-purin-9-yl]-2,3-dihydroxy-cyclopentyl}-propionamide;
N-{(1S,2R,3S,4R)-4-[2-Chloro-6-(4-phenylsulfanyl-piperidin-1-ylamino)-purin-9-yl]-2,3-dihydroxy-cyclopentyl}-propionamide;
N-[(1S,2R,3S,4R)-4-(2-[(1E)-3-[(Phenylamino)carbonyl]-1-triazenyl]-6-cyclopentylamino-purin-9-yl)-2,3-dihydroxy-cyclopentyl]-propionamide;
1-[6-((1R,2S,4S)-Bicyclo[2.2.1]hept-2-ylamino)-9-((1R,2S,3R,4S)-2,3-dihydroxy-4-propionylamino-cyclopentyl)-9H-purin-2-yl]-1H-pyrazole-4-carboxylic acid;
1-{9-((1R,2S,3R,4S)-2,3-Dihydroxy-4-propionylamino-cyclopentyl)-6-[(R)-(tetrahydro-furan-3-yl)amino]-9H-purin-2-yl}-1H-pyrazole-4-carboxylic acid amide;
   and pharmaceutically acceptable salts thereof.

In another embodiment, the present invention provides compounds of formula (I) independently selected from:
N-[(1S,2R,3S,4R)-4-(6-Cyclopentylamino-purin-9-yl)-2,3-dihydroxy-cyclopentyl]-propionamide;
N-[(1S,2R,3S,4R)-4-(2-Chloro-6-cyclopentylamino-purin-9-yl)-2,3-dihydroxy-cyclopentyl]-propionamide;
N-[(1S,2R,3S,4R)-4-(6-Cyclopentylamino-purin-9-yl)-2,3-dihydroxy-cyclopentyl]-2-hydroxy-acetamide;
N-[(1S,2R,3S,4R)-4-(2-Chloro-6-cyclopentylamino-purin-9-yl)-2,3-dihydroxy-cyclopentyl]-2-hydroxy-acetamide;
1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride;
N-{(1S,2R,3S,4R)-2,3-Dihydroxy-4-[6-((S)-2-methoxy-cyclopentylamino)-purin-9-yl]-cyclopentyl}-propionamide;
N-{(1S,2R,3S,4R)-2,3-Dihydroxy-4-[6-((1R,2S,4S)-bicyclo[2.2.1]heptan-2-amino)-purin-9-yl]-cyclopentyl}-propionamide;
(1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol;
1-[9-[(1R,2S,3R,4S)-2,3-Dihydroxy-4-(2-hydroxy-acetylamino)-cyclopentyl]-6-(3fluoro-4-hydroxy-phenylamino)-9H-purin-2-yl]-1H-pyrazole-4-carboxylic acid amide;
N-((1S,2R,3S,4R)-4-{6-[(R)-1-(3-Chloro-thiophen-2-ylmethyl)-propylamino)-purin-9-yl]-2,3-dihydroxy-cyclopentyl}-acetamide;
N-((1S,2R,3S,4R)-4-{6-[(R)-1-(4-Chloro-thiophen-3-ylmethyl)-propylamino)-purin-9-yl]-2,3-dihydroxy-cyclopentyl}-propionamide;
N-((1S,2R,3S,4R)-2,3-Dihydroxy-4-{6-[(R)-(tetrahydro-furan-3-yl)amino]-purin-9-yl}-cyclopentyl)-propionamide;
N-{(1S,2R,3S,4R)-2,3-Dihydroxy-4-[6-((1S,2S)-2-hydroxy-cyclopentylamino)-purin-9-yl]-cyclopentyl}-propionamide;
1-[6-((1R,2S,4S)-Bicyclo[2.2.1]hept-2-ylamino)-9-((1R,2S,3R,4S)-2,3-dihydroxy-4-propionylamino-cyclopentyl)-9H-purin-2-yl]-1H-pyrazole-4-carboxylic acid;
1-{9-((1R,2S,3R,4S)-2,3-Dihydroxy-4-propionylamino-cyclopentyl)-6-[(R)-(tetrahydro-furan-3-yl)amino]-9H-purin-2-yl}-1H-pyrazole-4-carboxylic acid amide;
   and pharmaceutically acceptable salts thereof.

### DEFINITIONS

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine.

“Hydroxy”, as used herein, is OH.

“C₁-C₈-alkyl”, as used herein, denotes straight chain or branched alkyl having 1 to 8 carbon atoms. Preferably C₁-C₈-alkyl is C₁-C₄-alkyl, specifically methyl, ethyl, n-propyl, i-propyl, n-butyl, t-butyl.

“C₁-C₈-alkoxy”, or as used herein, denotes straight chain or branched alkoxy having 1 to 8 carbon atoms, e.g., O—C₁-C₈-alkyl. Preferably, C₁-C₈-alkoxy is C₁-C₄-alkoxy.

“C₁-C₈-alkylamino” and “di-C₁-C₈-alkyl-amino”, (for example, —NHR⁶, —N(R⁶)₂ or NR³R⁴ when R³, R⁴, or R⁶, are alkyl groups) as used herein, denote amino substituted respectively by one or two C₁-C₈-alkyl groups as hereinbefore defined, which may be the same or different.

“C₁-C₈-alkylcarbonyl” and “C₁-C₈-alkoxycarbonyl”, (for example, the C(O)OR², or R¹ portion of —NHC(O)OR² or —NHC(O)OR¹ when R¹ or R² are alkyl or alkoxygroups) as used herein, denote C₁-C₈-alkyl or C₁-C₈-alkoxy, respectively, as hereinbefore defined attached by a carbon atom to a carbonyl group.

“aryl”, as used herein, means a “(C₆-C₁₀)aryl” group and, denotes a monovalent carbocyclic aromatic group that contains 6 to 10 carbon atoms and which may be, e.g., a monocyclic group, such as phenyl; or a bicyclic group, such as naphthyl. Preferably “aryl” is phenyl.

“C₇-C₁₄-aralkyl”, as used herein, denotes alkyl, e.g., C₁-C₄-alkyl, as hereinbefore defined, substituted by C₆-C₁₀-aryl as hereinbefore defined (for example, the R⁶(aryl) portion of —NHR⁶(aryl) when R⁶ is an alkyl group). Preferably, C₇-C₁₄-aralkyl is C₇-C₁₀-aralkyl, such as phenyl-C₁-C₄-alkyl.

“C₁-C₈-alkylaminocarbonyl” and “C₃-C₈-cycloalkylaminocarbonyl” as used herein denote C₁-C₈-alkylamino and C₃-C₈-cycloalkylamino respectively as hereinbefore defined attached by a carbon atom to a carbonyl group. Preferably C₁-C₈-alkylaminocarbonyl and C₃-C₈-cycloalkyl-aminocarbonyl are C₁-C₄-alkylaminocarbonyl and C₃-C₈-cycloalkylaminocarbonyl, respectively.

“Heteroaryl” refers to an aromatic group of from 2 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“4- to 8-membered heterocyclic ring containing at least one ring heteroatom selected from the group including nitrogen, oxygen and sulfur”, and may optionally be benzo-fused, as used herein, may be, e.g., furan, pyrrole, pyrrolidine, pyrazole, imidazole, triazole, thiazole, benzothiazole, thiophene, triazine, isotriazole, tetrazole, thiadiazole, isothiazole, oxadiazole, pyridine, piperidine, oxazole, isoxazole, pyrazine, pyridazine, pyrimidine, piperazine, pyrrolidine, morpholino, triazine, oxazine or thiazole. Preferred heterocyclic rings include pyrazole, tetrazole, triazole, pyridine, furan, thiophene, triazine, tetrahydropyran, benzothiazole and pyran. The 4-to-8-membered heterocyclic ring can be unsubstituted or substituted.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps. As understood by one skilled in the art only combinations of substituents that are chemically possible are embodiments of the invention.

All combinations of variables as defined anywhere above are considered to be within the scope of the invention. Thus, the invention comprises compounds in which X is as defined anywhere herein, Y is as defined anywhere herein and Z is as defined anywhere herein.

Suitable specific compounds of formula (I) or (Ia) are those described hereinafter in the Examples.

Compounds of the invention (i.e. compounds of formula (I) or (Ia)) that contain a basic centre are capable of forming acid addition salts, particularly pharmaceutically acceptable acid addition salts. Pharmaceutically acceptable acid addition salts of the compounds of the invention include those of inorganic acids, for example, hydrohalic acids such as hydrofluoric acid, hydrochloric acid, hydrobromic acid, hydroiodic acid, nitric acid, sulfuric acid, phosphoric acid; and organic acids, for example aliphatic monocarboxylic acids such as formic acid, acetic acid, trifluoroacetic acid, propionic acid and butyric acid, caprylic acid, dichloroacetic acid, hippuric acid, aliphatic hydroxy acids such as lactic acid, citric acid, tartaric acid or malic acid, gluconic acid, mandelic acid, dicarboxylic acids such as maleic acid or succinic acid, adipic acid, aspartic acid, fumaric acid, glutamic acid, malonic acid, sebacic acid, aromatic carboxylic acids such as benzoic acid, p-chloro-benzoic acid, nicotinic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid, ethanesulfonic acid, ethane-1,2-disulfonic acid, 2-hydroxy-ethanesulfonic acid, (+) camphor-10-sulfonic acid, naphthalene-2-sulfonic acid, naphthalene-1,5-disulfonic acid or p-toluenesulfonic acid. These salts may be prepared from compounds of the invention by known salt-forming procedures. Pharmaceutically acceptable solvates are generally hydrates.

Compounds of the invention which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine, arginine, benethamine, benzathine, diethanolamine, 4-(2-hydroxy-ethyl)morpholine, 1-(2-hydroxyethyl)pyrrolidine, N-methyl glutamine, piperazine, triethanol-amine or tromethamine. These salts may be prepared from compounds of the invention by known salt-forming procedures. Compounds of the invention that contain acidic, e.g. carboxyl, groups may also exist as zwitterions with the quaternary ammonium centre.

Compounds of the invention in free form may be converted into salt form, and vice versa, in a conventional manner. The compounds in free or salt form can be obtained in the form of hydrates or solvates containing a solvent used for crystallisation. Compounds of the invention can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Some compounds of the invention contain at least one asymmetric carbon atom and thus they exist in individual optically active isomeric forms or as mixtures thereof, e.g. as racemic mixtures. In cases where additional asymmetric centres exist the present invention also embraces both individual optically active isomers as well as mixtures, e.g. diastereomeric mixtures, thereof.

The invention includes all such forms, in particular the pure isomeric forms. The different isomeric forms may be separated or resolved one from the other by conventional methods, or any given isomer may be obtained by conventional synthetic methods or; by stereospecific or asymmetric syntheses. Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

The invention includes all pharmaceutically acceptable isotopically-labelled compounds of the invention wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g. ²H and ³H, carbon e.g. ¹¹C, ¹³C and ¹⁴C, chlorine e.g. ³⁶Cl, fluorine e.g. ¹⁸F, iodine e.g. ¹²³I and ¹²⁵I, nitrogen e.g. ¹³N and ¹⁵N, oxygen e.g. ¹⁵O, ¹⁷O and ¹⁸O, and sulfur e.g. ³⁵S.

Certain isotopically-labelled compounds of the invention, for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be useful in Positron Emission Topography (PET) studies for examining substrate receptor occupancy.

Isotopically-labelled compounds of the invention can generally be prepared by conventional techniques known to those skilled in the art or by processes analogous to those described in the accompanying examples using an appropriate isotopically-labelled reagent in place of the non-labelled reagent previously used.

Pharmaceutically acceptable solvates in accordance with the invention include those wherein the solvent of crystallisation may be isotopically substituted e.g. D₂O, d₆-acetone or d₆-DMSO.

The compounds of the invention may exist in both unsolvated and solvated forms. The term “solvate” is used herein to describe a molecular complex comprising the compound of the invention and one or more pharmaceutically acceptable solvent molecules, e.g., ethanol. The term “hydrate” is employed when said solvent is water.

### Synthesis:

Described below general routes for the preparation of compounds of formula (I).

Scheme 1 illustrates a synthetic route for the preparation of compounds of formula (I), where R¹ and R^{A} are as defined hereinbefore from an intermediate compound A (wherein P=benzyl). This route may also be utilitsed when starting from analogues of compound A (wherein P=t-butyl). When X=benzyl, deprotection through hydrogenolysis can also remove the 2-chloro substituent to deliver compounds with a hydrogen atom at the 2-position.

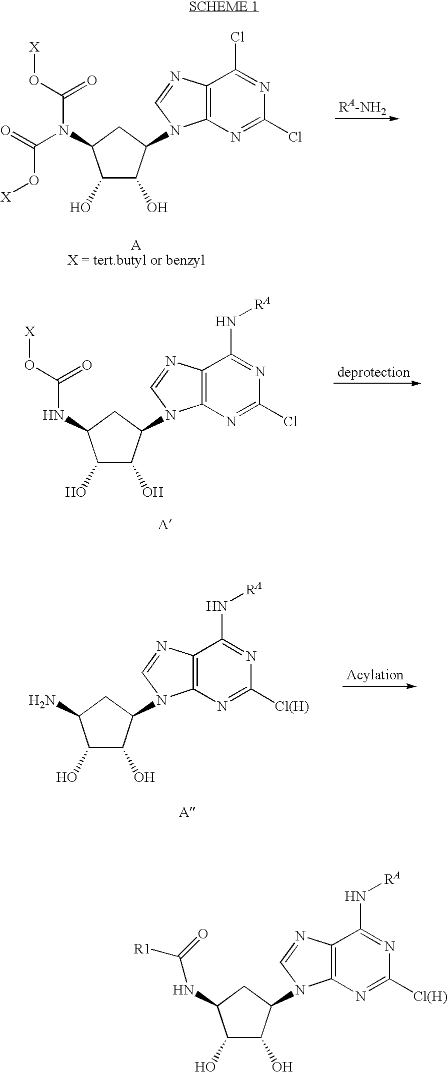

According to a further aspect the present invention provides a process for the preparation of compounds of formula (IA) essentially as illustrated in Scheme 1 comprising:
(a) reaction of intermediate A, or the t-butyl analogue thereof, with a compound of formula R^{A}—NH₂ to provide an intermediate compound A′;
(b) deprotection of intermediate compound A′ to provide intermediate compound A″; and
(c) acylation of intermediate compound A″ to provide a compound of formula (IA).
The present invention additionally, and independently, provides intermediate compounds of formulae (A), (A′), and (A″).
Scheme 2 illustrates a synthetic route for the preparation of further compounds of formula (I) with alternative 2-substiuents, where R¹ and R^{A} are as defined hereinbefore from an intermediate compound A (wherein X=benzyl)

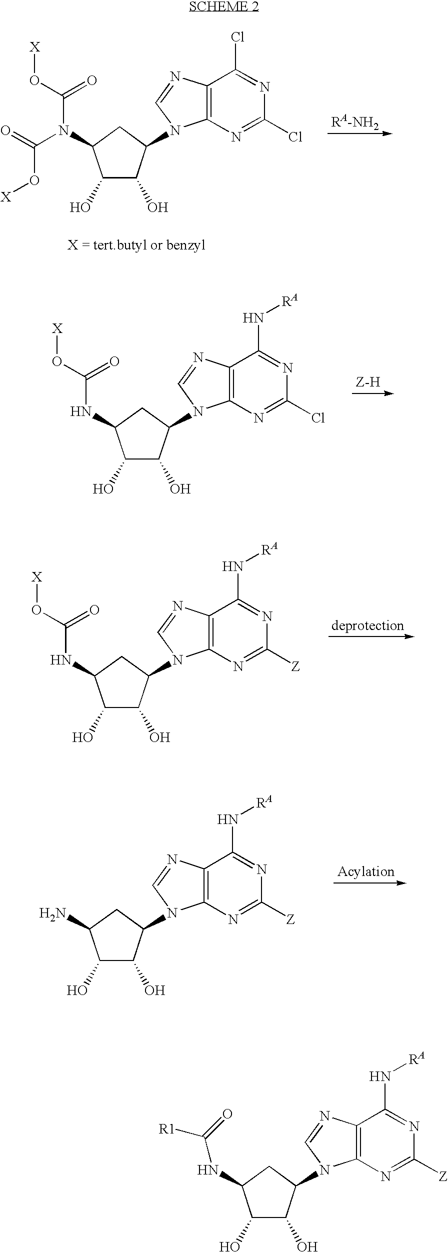

The Invention also provides, in another aspect, a method of preparing a compound of formula (I), in free or salt form which comprises:
(i) (A) for the preparation of compounds of formula (I), reacting a compound of formula (Ib)

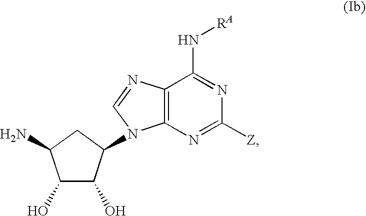

where Z and R^{A} are as hereinbefore defined, with acetyl chloride in the presence of base;
(B) for the preparation of compounds of formula (I), (i) reacting a compound of formula (Ic)

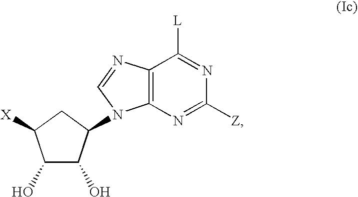

where
   X and Z are as hereinbefore defined; and
   L is a leaving group, with a compound of formula H₂N—R^{A}, where R^{A} is as hereinbefore defined in the presence of a base; and
(ii) recovering the resultant compound of formula (I), in free or pharmaceutically acceptable salt form.
   (C) for the preparation of compounds of formula (I), (i) reacting a compound of formula (Id)

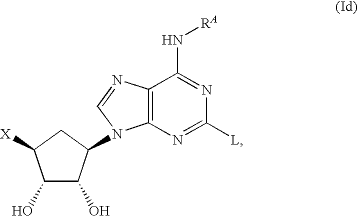

wherein
   X and R^{A} are as hereinbefore defined; and
   L is a leaving group, with a compound of formula Z—H, where Z is as hereinbefore defined in the presence of a base; and
(ii) recovering the resultant compound of formula (I), in free or pharmaceutically acceptable salt form.

The compounds of formula (I) can be prepared, e.g., using the general reactions and techniques described hereinbefore and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Protective Groups In Organic Synthesis, Greene and Wuts, Eds., Wiley and Sons (1999). It is understood by those skilled in the art that only combinations of substituents that are chemically possible are embodiments of the present invention.

Compounds of formulae (I) and (IA), in free form, may be converted into salt form, and vice versa, in a conventional manner. The compounds in free or salt form can be obtained in the form of hydrates or solvates containing a solvent used for crystallisation. Compounds of formulae (I) and (IA) can be recovered from reaction mixtures and purified in a conventional manner. Isomers, such as stereoisomers, may be obtained in a conventional manner, e.g. by fractional crystallisation or asymmetric synthesis from correspondingly asymmetrically substituted, e.g. optically active, starting materials.

In those compounds where there is an asymmetric carbon atom the compounds exist in individual optically active isomeric forms or as mixtures thereof, e.g. as racemic or diastereomeric mixtures. The present invention embraces both individual optically active R and S isomers as well as mixtures, e.g. racemic or diastereomeric mixtures, thereof.

Pharmaceutically acceptable salts of the compound of formula (I) may be acid or base addition salts, including those of inorganic acids, for example hydrohalic acids such as hydrofluoric acid, hydrochloric acid, hydrobromic acid or hydroiodic acid; nitric acid, sulfuric acid, phosphoric acid; and organic acids such as formic acid, acetic acid, propionic acid, butyric acid, benzoic acid, o-hydroxybenzoic acid, p-hydroxybenzoic acid, p-chlorobenzoic acid, diphenylacetic acid, triphenylacetic acid, 1-hydroxynaphthalene-2-carboxylic acid, 3-hydroxynaphthalene-2-carboxylic acid, aliphatic hydroxy acids such as lactic acid, citric acid, tartaric acid or malic acid, dicarboxylic acids such as fumaric acid, maleic acid or succinic acid, and sulfonic acids such as methanesulfonic acid or benzenesulfonic acid. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

These salts may be prepared from compounds of formula (I) by known salt-forming techniques. Pharmaceutically acceptable salts are generally hydrates. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

### Pharmacological Activity and Use

Compounds of formula (I) and their pharmaceutically acceptable salts are useful as pharmaceuticals. In particular, they are adensosine receptor ligands, in particular as adenosine A₁ receptor agonists. Diseases that can be treated using the method of this invention include, but are not limited to, insomnia, sleep apnoea, supraventricular tachycardia incuding atrial fibrillation and atrial flutter, congestive heart failure, stroke, diabetes, obesity, epilepsy, ischemia, stable angina, unstable angina, irritable bowel syndrome, nausea and myocardial infraction. The method of the invention is also useful in treating hyperlipidemic conditions, and is therefore useful in treating metabolic disorders, including type II diabetes, hypertriglyceridemia and metabolic syndrome. The method of the invention are also useful in protecting tissues being maintained for transplantation. The method of the invention are also useful as analgesics for relieving pain in conditions including, but not limited to, neuropathic conditions such as fibomyalgia and post herpetic neuralgia, rheumatoid arthritis, osteaoarthritis, trigeminal neuralgia, neuropathies associated with cancer, and pain associated with migrane, tension headache, cluster headaches, functional bowel disorders, non cardiac chest pain and non ulcer dyspepsia. The method of the invention are also useful as CNS agents, e.g. as hypnotics, sedatives, analgesics and anti-convulsants.

Compounds of the present invention have pEC₅₀ values as agonists below 1.0.×10⁻⁶ in the following assay.

In Vitro Functional Activity at the Human a Receptor Based on the Stimulation of [³⁵S]-GTPγS Binding

In brief the assay is based on the conventional GTPγS binding assay described by ([Lorenzen A, Guerra L, Vogt H, et al, (1996)] Interaction of full and partial agonists of the A₁ adenosine receptor with receptor/G protein complexes in rat brain membranes. Mol Pharmacol. 49(5):915-26) The assay is run as a SPA assay where the A₁ membranes are captured by wheatgerm agglutinin (WGA) SPA beads, through a specific interaction between WGA and carbohydrate residues of glycoprotein's on the surfaces for the membranes. Upon receptor stimulation, [³⁵S]-GTPγS binds specifically to the alpha subunit of the G-protein thus bringing the [³⁵S]-GTPγS into close proximity with the SPA beads. Emitted β particles from the [³⁵S]-GTPγS excite the scintillant in the beads and produce light. Free [³⁵S]-GTPγS in solution is not in close proximity to the SPA beads and therefore does not activate the scintillant and hence does not produce light. The assays were performed in a final volume of 250 μL per well in a white non-binding surface 96-well Optiplates and could be run in either an agonist format, or an antagonist format (pre-incubation with an EC₅₀ concentration of the appropriate receptor agonist).

Preferred compounds of the invention have pEC₅₀ values below 1.0×10⁻⁷ in said assay.

### A1 Assay Protocol

### LIST OF ABBREVIATIONS

| | |
|---|---|
| A₃ | Adenosine A₃ receptor |
| BSA | Bovine serum albumin |
| CHO | Chinese hamster ovary |
| DMSO | Dimethyl sulphoxide |
| EDTA | Ehylenediaminetetraacetic acid |
| FCS | Fetal calf serum |
| HEPES | 4-(2-Hydroxyethyl)piperazine-1- |
| | ethanesulfonic acid |
| I-AB-MECA | N6-(4-Amino-3-iodobenzyl)-5′- |
| | N-methylcarbamoyl-adenosine |
| K_{d} | Dissociation constant |
| MgCl₂ | Magnesium chloride |
| NaCl | Sodium chloride |
| Tris-HCl | Tris(hydroxymethyl)- |
| | aminomethane hydrochloride |

### Introduction

Adenosine, an endogenous modulator of a wide range of biological functions, interacts with at least four cell surface receptor subtypes classified as A₁, A_{2A}, A_{2B} and A₃, all of which are coupled to G proteins. See Linden, Annu Rev Pharmacol Toxicol, Vol. 41, pp. 775-787 (2001) and Jacobsen and Gao, Nature Reviews Drug Discovery, Vol. 5, pp.: 247-264 (2006).

Accordingly, agents of the invention can be useful for the treatment of a condition mediated by activation of the adenosine A₁ receptor.

For instance, the compounds of the present invention can used to treat treatment of diseases such as type-2 diabetes, arrhythmia, pain and insomnia. Preferably, the compounds of the present invention are used for the treatment of type-2 diabetes, pain and sleep disorders.

The utility of adenosine A₁ receptor agonisits in the treatment of sleep disorders has been highlighted in the following references: Blanco-Centurion et al, Adenosine and sleep homeostasis in the basal forebrain, Journal of Neuroscience (2006), 26(31), 8092-8100. Marks et al, Adenosine A1 receptors mediate inhibition of cAMP formation in vitro in the pontine, REM sleep induction zone, Brain Research (2005), 1061(2), 124-127. Thakkar et al, Adenosinergic inhibition of basal forebrain wakefulness-active neurons: a simultaneous unit recording and microdialysis study in freely behaving cats Neuroscience (2003), 122(4), 1107-1113.

The present invention concerns, by one embodiment, a method for the treatment of pain, sleep disorders and/or type-2 diabetes in a human subject, comprising administering to an individual in need of such treatment an effective amount of an A₃RAg.

The agonist according to the invention is either a full or partial agonist of the adenosine A₁ receptor. As used herein, a compound is a “full agonist” of an adenosine A₁ receptor if it is able to fully inhibit adenylate cyclase, a compound is a “partial agonist” of an adenosine A₁ receptor if it is able to partially inhibit adenylate cyclase.

The method of the present invention can have particular usefulness in vivo.

The agents of the invention may be administered by any appropriate route, e.g., orally, e.g., in the form of a tablet or capsule; parenterally, e.g., intravenously; by inhalation, or as described in WO 01/23399, WO 95/02604, WO 05/063246, WO 02/055085 and WO 06/011130. Preferably the agents of the invention are administered by the oral, intranasal, inhaled or sublingual route, and more preferably via the oral route.

In a further aspect, the invention also provides a pharmaceutical composition comprising a compound of formula (I), in free form or in the form of a pharmaceutically acceptable salt, optionally together with a pharmaceutically acceptable diluent or carrier therefor. The composition may contain a co-therapeutic agent. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations. Other formulations can be as described in WO 01/23399, WO 95/02604, WO 05/063246, WO 02/055085 and WO 06/011130.

Dosages of compounds of formula (I) employed in practising the present invention will of course vary depending, e.g., on the particular condition to be treated, the effect desired and the mode of administration as described in WO 01/23399, WO 95/02604, WO 05/063246, WO 02/055085 and WO 06/011130.

The invention is illustrated by the following Examples of Compounds of Formula I.

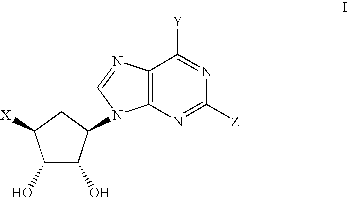

Examples 1-34 are illustrated in Table 1 below. Methods for preparing such compounds are described hereinafter.

TABLE 1

| Ex. | X | Y | Z |
|---|---|---|---|
| 1 | 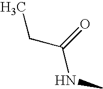 | 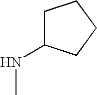 | —H |
| 2 | 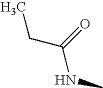 | 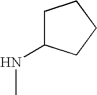 | —Cl |
| 3 | 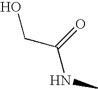 | 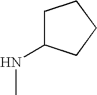 | —H |
| 4 | 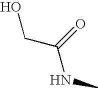 | 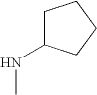 | —Cl |
| 5 | 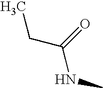 | 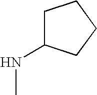 | 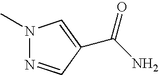 |
| 6 | 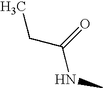 | 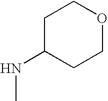 | —H |
| 7 | 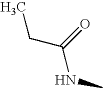 | 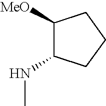 | —H |
| 8 | 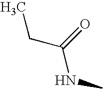 | 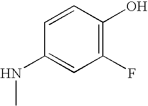 | —H |
| 9 | 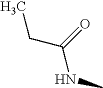 | 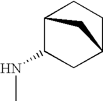 | —H |
| 10 | 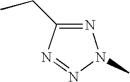 | 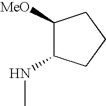 | —Cl |
| 11 | 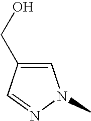 | 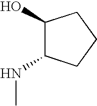 | —H |
| 12 | 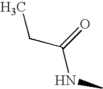 | 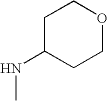 | 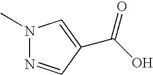 |
| 13 | 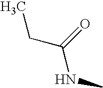 | 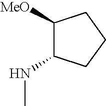 | 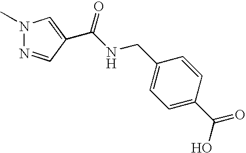 |
| 14 | 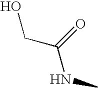 | 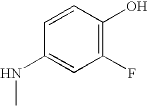 | 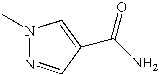 |
| 15 | 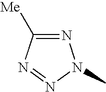 | 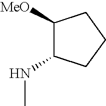 | 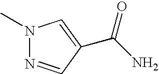 |
| 16 | 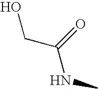 | 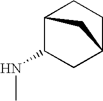 | 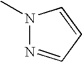 |
| 17 | 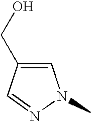 | 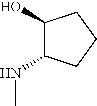 | 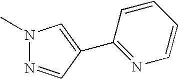 |
| 18 | 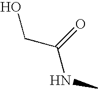 | 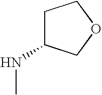 | 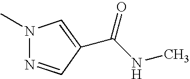 |
| 19 | 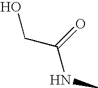 | 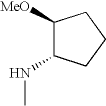 | 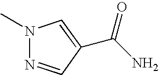 |
| 20 | 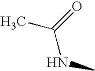 | 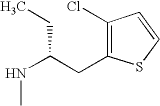 | —H |
| 21 | 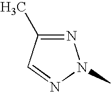 | 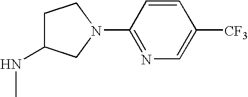 | —H |
| 22 | 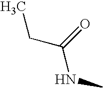 | 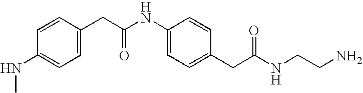 | —H |
| 23 | 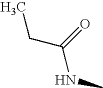 | 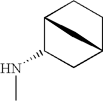 | —H |
| 24 | 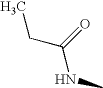 | 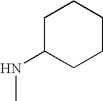 | —H |
| 25 | 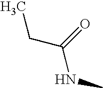 | 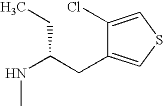 | —H |
| 26 | 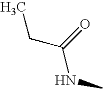 | 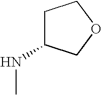 | —H |
| 27 | 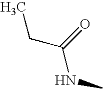 | 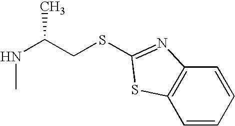 | —Cl |
| 28 | 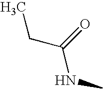 | 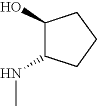 | —H |
| 29 | 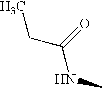 | 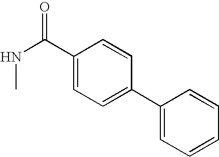 | —H |
| 30 | 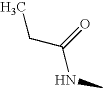 | 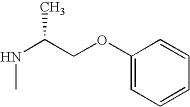 | —Cl |
| 31 | 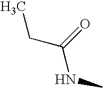 | 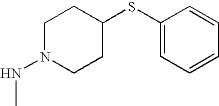 | —Cl |
| 32 | 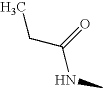 | 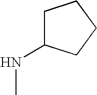 | 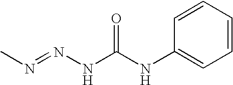 |
| 33 | 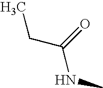 | 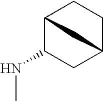 | 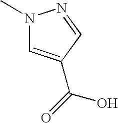 |
| 34 | 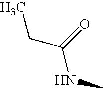 | 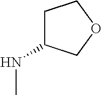 | 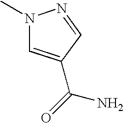 |

In the Experimental Section the following abbreviations have been used:

RT room temperature
DMF dimethyl-formamide
DIPEA diisopropylethylamine

### NMP N-methylpyrrolidine

THF tetrahydrofuran
MeOH methanol
DCM dichloromethane
EtOAc ethyl acetate
EtOH ethanol
LCMS liquid chromatographic mass spectroscopy
TEA triethylamine.

### HPLC High Performance Liquid Chromatography

### HCl Hydrochloric Acid

The following standard chemical reagents within the common general knowledge of the skilled chemist have been utilized: Hunig's base. Methods of preparation of such compounds are well-known.

In addition various trade reagents and materials available from have been utilized. Such reagents and materials include: Isolute™ (available from Biotage), and can be readily obtained from the suppliers indicated.

Mass spectra are run on open access LCMS systems using electrospray ionization. These are either Agilent 1100 HPLC/Micromass Platform Mass Spectrometer combinations or Waters Acquity UPLC with SQD Mass Spectrometer. [M+H]+ refers to mono-isotopic molecular weights.

NMR spectra are run on open access Bruker AVANCE 400 NMR spectrometers using ICON-NMR. Spectra are measured at 298K and are referenced using the solvent peak.

### EXAMPLE 1

### N-[(1S,2R,3S,4R)-4-(6-Cyclopentylamino-purin-9-yl)-2,3-dihydroxy-cyclopentyl]-propionamide

A stirred solution of (1S,2R,3S,5R)-3-amino-5-(6-cyclopentylamino-purin-9-yl)-cyclopentane-1,2-diol hydrochloride (Intermediate D) (102 mg) in DMF (1 ml) is treated with DIPEA (250 μl) at room temperature. The resulting suspension is treated with propionyl chloride (25 μl) and stirred at RT for 18 hours. Purification of the resulting mixture by reverse phase column chromatography (Isolute™ C18, 0-100% acetonitrile in water −0.1% HCl) affords the title compound as a white glassy solid. [M+H]⁺ 375.

### EXAMPLE 2

### N-[(1S,2R,3S,4R)-4-(2-Chloro-6-cyclopentylamino-purin-9-yl)-2,3-dihydroxy-cyclopentyl]-propionamide

This compound is prepared analogously to Example 1 by replacing (1S,2R,3S,5R)-3-amino-5-(6-cyclopentylamino-purin-9-yl)-cyclopentane-1,2-diol hydrochloride (Intermediate D) with (1S,2R,3S,5R)-3-amino-5-(2-chloro-6-cyclopentylamino-purin-9-yl)-cyclopentane-1,2-diol (Intermediate E) to afford the title compound as an off-white solid. [M+H]⁺ 409 and 411.

### EXAMPLE 3

### N-[(1S,2R,3S,4R)-4-(6-Cyclopentylamino-purin-9-yl)-2,3-dihydroxy-cyclopentyl]-2-hydroxy-acetamide

DIPEA (147 μl) is added to a solution of (1S,2R,3S,5R)-3-amino-5-(6-cyclopentylamino-purin-9-yl)-cyclopentane-1,2-diol hydrochloride (Intermediate D) (60 mg) in DMF (0.5 ml) at RT and stirred for 2 minutes. The resulting suspension is treated with acetoxyacetyl chloride (18 μl) and stirred at RT for 18 hours. MeOH (1 ml) is then added to the mixture followed by potassium carbonate (120 mg) and stirring continued for 18 hours at RT. The mixture is diluted with water to limit solubility and purification by reverse phase column chromatography (Isolute™ C18, 0-100% acetonitrile in water −0.1% HCl) affords the title compound as a colourless glassy solid. [M+H]⁺ 377.

### EXAMPLE 4

### N-[(1S,2R,3S,4R)-4-(2-Chloro-6-cyclopentylamino-purin-9-yl)-2,3-dihydroxy-cyclopentyl]-2-hydroxy-acetamide

This compound is prepared analogously to Example 3 by replacing (1S,2R,3S,5R)-3-amino-5-(6-cyclopentylamino-purin-9-yl)-cyclopentane-1,2-diol hydrochloride (Intermediate D) with (1S,2R,3S,5R)-3-amino-5-(2-chloro-6-cyclopentylamino-purin-9-yl)-cyclopentane-1,2-diol (Intermediate E) to afford the title compound as a white amorphous solid. [M+H]⁺ 411 and 413.

### EXAMPLE 5

### 1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride

### Step 1: 1-[9-((1R,2S,3R,4S)-4-Amino-2,3-dihydroxy-cyclopentyl)-6-cyclopentylamino-9H-purin-2-yl]-1H-pyrazole-4-carboxylic acid amide hydrochloride

A solution of (1S,2R,3S,4R)-4-(2-chloro-6-(cyclopentylamino)-9H-purin-9-yl)-2,3-dihydroxycyclopentylcarbamate (Intermediate C) (200 mg) and 1H-pyrazole-4-carboxylic acid amide (Intermediate F) (183 mg) in NMP (822 μl) is treated with potassium carbonate (284 mg) and sealed under an atmosphere of argon. The mixture is heated using microwave radiation at 180° C. for 30 minutes and then diluted with aqueous HCl. Purification of the crude mixture by reverse phase column chromatography (Isolute™ C18, 0-100% acetonitrile in water −0.1% HCl) affords the title compound as a white solid. [M+H]⁺ 428.

Step 2: 1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride This compound is prepared analogously to Example 1 by replacing (1S,2R,3S,5R)-3-amino-5-(6-cyclopentylamino-purin-9-yl)-cyclopentane-1,2-diol hydrochloride (Intermediate D) with 1-[9-((1R,2S,3R,4S)-4-amino-2,3-dihydroxy-cyclopentyl)-6-cyclopentylamino-9H-purin-2-yl]-1H-pyrazole-4-carboxylic acid amide hydrochloride (Example 5 step 1) to afford a white amorphous solid. [M+H]⁺ 484.

### EXAMPLES 6-9

These compounds namely,
N-{(1S,2R,3S,4R)-2,3-Dihydroxy-4-[6-(tetrahydro-pyran-4-ylamino)-purin-9-yl]-cyclopentyl}-propionamide (Ex 6),
N-{(1S,2R,3S,4R)-2,3-Dihydroxy-4-[6-((S)-2-methoxy-cyclopentylamino)-purin-9-yl]-cyclopentyl}-propionamide (Ex.7),
N-{(1S,2R,3S,4R)-4-[6-(3-Fluoro-4-hydroxy-phenylamino)-purin-9-yl]-2,3-dihydroxy-cyclopentyl}-propionamide (Ex.8) and
N-{(1S,2R,3S,4R)-2,3-Dihydroxy-4-[6-((1R,2S,4S)-bicyclo[2.2.1]heptan-2-amino)-purin-9-yl]-cyclopentyl}-propionamide (Ex.9),
   are prepared from Intermediates B and C analogously to Example 1 by replacing cyclopentylamine with the following amines: (Ex 6) tetrahydro-2H-pyran-4-amine; (Ex 7) (1S,2S)-2-methoxycyclopentylamine, prepared according to the procedure as described in WO 2002/074780, at page 39, Example 24; (Ex 8) 4-amino-2-fluorophenol; and (Ex 8) (1R,2S,4S)-bicyclo[2.2.1]heptan-2-amine, prepared according to the procedure described in EP 2911051 page 10 preparation VI.

### EXAMPLE 10

### (1R,2S,3R,5S)-3-[2-Chloro-6-[(1S,2S)-2-methoxycyclopentylamino]-purin-9-yl]-5-(5-ethyl-tetrazol-2-yl)-cyclopentane-1,2-diol

### Step 1: 2,6-Dichloro-9-[(1R,4S)-4-(5-ethyl-tetrazol-2-yl)-cyclopent-2-enyl]-9H-purine

Carbonic acid (1S,4R)-4-(2,6-dichloro-purin-9-yl)-cyclopent-2-enyl ester ethyl ester (prepared according to the procedure as described on page 37, Intermediate AC of, WO 2006/074925) (3 g, 8.75 mmol), 5-Ethyl-2H-tetrazole (0.94 g. 9.62 mmol), tris(dibenzylideneacetone)dipalladium(0) (0.40 g, 0.44 mmol) and triphenylphosphine (0.35 g, 1.32 mmol) are placed in an oven-dried flask under an atmosphere of argon. Dry deoxygenated THF (40 ml) is added and the reaction mixture is stirred gently for 5 minutes at RT. Triethylamine (20 ml) is added and the reaction mixture is stirred at RT for 1 hour. The solvent is removed in vacuo, the residue taken up in MeOH (50 ml), and the title compound collected by filtration. ¹H nmr (CDCl₃, 400 MHz); 8.55 (s, 1H), 6.35 (m, 1H), 6.25 (m, 1H), 6.05 (m, 1H), 5.90 (m, 1H), 3.45 (m, 1H), 2.85 (q, 2H), 2.30 (m, 1H), 1.30 (t, 3H), [M+H]⁺ 351.

### Step 2: {2-Chloro-9-[(1R,4S)-4-(5-ethyl-tetrazol-2-yl)-cyclopent-2-enyl]-9H-purin-6-yl}-[(1S,2S)-2-methoxycyclopentyl]-amine

2,6-Dichloro-9-[(1R,4S)-4-(5-ethyl-tetrazol-2-yl)-cyclopent-2-enyl]-9H-purine (Step 1) is dissolved in THF under an atmosphere of argon. (1S,2S)-2-methoxycyclopentylamine (prepared according to the procedure illustrated at page 39, Example 24 of WO 2002/074780) is added and the reaction mixture is stirred at 50° C. for 4 hours. The solvent is removed in vacuo and residue is partitioned between dichloromethane and 2M HCl. The organic layer is washed with saturated NaHCO₃, water and brine, dried over MgSO₄, filtered and the solvent is removed in vacuo to give the title compound.

### Step 3: (1R,2S,3R,5S)-3-[2-Chloro-6-[(1S,2S)-2-methoxycyclopentylamino]-purin-9-yl]-5-(5-ethyl-tetrazol-2-yl)-cyclopentane-1,2-diol

{2-Chloro-9-[(1R,4S)-4-(5-ethyl-tetrazol-2-yl)-cyclopent-2-enyl]-9H-purin-6-yl}-[(1S,2S)-2-methoxycyclopentyl]-amine (Step 2) is dissolved in THF N-methylmorpholine N-oxide is added followed by osmium tetroxide. The reaction mixture is stirred at RT until complete. The solvent is removed in vacuo and the title compound is obtained after purification by reverse phase column chromatography.

### EXAMPLE 11

(1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol

### Step 1: {1-[(1S,4R)-4-(2,6-Dichloro-purin-9-yl)-cyclopent-2-enyl]-1H-pyrazol-4-yl}-methanol

A stirred mixture comprising carbonic acid, (1S,4R)-4-(2,6-dichloro-purin-9-yl)-cyclopent-2-enyl ester ethyl ester, prepared according to the procedure illustrated at page 37, Intermediate AC of WO 2006/074925, (1.00 g, 2.92 mmol), (1H-pyrazol-4-yl)-methanol (Intermediate G) (0.34 g, 3.50 mmol) and triphenyl phosphine (0.115 g, 0.44 mmol) in deoxygenated THF (10 ml) under an inert atmosphere of argon is treated with tris(dibenzylideneacetone)dipalladium (0) (0.13 g, 0.15 mmol) and then stirred at 50° C. for 1 hour. The solvent is removed in vacuo and the crude product is purified by chromatography on silica eluting with MeOH/DCM (1:25) to yield the title compound.

### Step 2: (1-{(1S,4R)-4-[2-Chloro-6-[(1S,2S)-2-hydroxycyclopentylamino]-purin-9-yl]-cyclopent-2-enyl}-1H-pyrazol-4-yl)-methanol

A mixture comprising {1-[(1S,4R)-4-(2,6-dichloro-purin-9-yl)-cyclopent-2-enyl]-1H-pyrazol-4-yl}-methanol (Step 1) and (1S,2S)-2-amino-cyclopentanol in dry THF is stirred at 35° C. for 3 days. The solvent is removed in vacuo and the resulting crude residue is partitioned between DCM and 0.1 M HCl. The organic portion is separated, washed with water, brine, dried (MgSO₄) and concentrated in vacuo to afford the title product.

### Step 3: (1R,2S,3R,5S)-3-[2-chloro-6-[(1S,2S)-2-hydroxycyclopentylamino]-purin-9-yl]-5-(4-hydroxymethyl-pyrazol-1-yl)-cyclopentane-1,2-diol

(1-{(1S,4R)-4-[2-Chloro-6-[(1S,2S)-2-hydroxycyclopentylamino]-purin-9-yl]-cyclopent-2-enyl}-1H-pyrazol-4-yl)-methanol (step 2) and 4-methylmorpholine-N-oxide in THF is treated with osmium tetroxide (2 ml of a 4% solution in water) and stirred at RT overnight. The solvent is removed in vacuo and the resulting crude residue is partitioned between DCM and 0.1 M HCl. The organic portion is dried (MgSO₄) and concentrated in vacuo to give the title product.

### Step 4: (1R,2S,3R,5S)-3-[6-(1S,2S)-2-hydroxycyclopentylamino]-purin-9-yl]-5-(4-hydroxymethyl-pyrazol-1-yl)-cyclopentane-1,2-diol

Hydrogenation of (1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol (Step 3) in an analogous manner to that used to prepare Intermediates D and E gives the title compound.

### EXAMPLE 12

### 1-[6-{tetrahydro-2H-pyran-4-amino}-9-((1R,2S,3R,4S)-2,3-dihydroxy-4-propionylamino-cyclopentyl)-9H-purin-2-yl]-1H-pyrazole-4-carboxylic acid tetrahydro-2H-pyran-4-amine

### Step 1: N-[(1S,2R,3S,4R)-4-(2-Chloro-6-[tetrahydro-2H-pyran-4-amino]-purin-9-yl)-2,3-dihydroxy-cyclopentyl]-propionamide

This compound is prepared from Intermediate A in an analogous sequence to that used to prepare Example 1 by replacing cyclopentylamine with tetrahydro-2H-pyran-4-amine.

### Step 2: N-[(1S,2R,3S,4R)-4-(6-[tetrahydro-2H-pyran-4-amino]-2-hydrazino-purin-9-yl)-2,3-dihydroxy-cyclopentyl]-propionamide

A mixture comprising N-[(1S,2R,3S,4R)-4-(2-Chloro-6-[tetrahydro-2H-pyran-4-amino]-purin-9-yl)-2,3-dihydroxy-cyclopentyl]-propionamide, and hydrazine mono-hydrate is stirred at RT for 72 h. Isopropyl alcohol is then added and the solvent was decanted off to afford a gummy mixture which is dissolved in water and stirred for 12 h. The fine solid obtained is filtered, washed with water and dried in vacuo to afford the title compound.

### Step 3: 1-[6-[tetrahydro-2H-pyran-4-amino]-9-((1R,2S,3R,4S)-2,3-dihydroxy-4-propionylamino-cyclopentyl)-9H-purin-2-yl]-1H-pyrazole-4-carboxylic acid ethyl ester

To a solution of N-[(1S,2R,3S,4R)-4-(6-[tetrahydro-2H-pyran-4-amino]-2-hydrazino-purin-9-yl)-2,3-dihydroxy-cyclopentyl]-propionamide in dry ethyl alcohol is added 2-formyl-3-oxo-propionic acid ethyl ester, prepared according to the process illustrated at page 2217, Intermediate 1 of Bertz S. H., Dabbagh G. and Cotte P.; J. Org. Chem. 1982, 47, 2216-2217. The reaction mixture is heated at reflux for 8 hours then concentrated in vacuo. The crude residue is purified by chromatography on silica eluting with MeOH in chloroform to afford the title compound.

### Step 4: 1-[6-{tetrahydro-2H-pyran-4-amino}-9-((1R,2S,3R,4S)-2,3-dihydroxy-4-propionylamino-cyclopentyl)-9H-purin-2-yl]-1H-pyrazole-4-carboxylic acid

Hydrolysis of N-[(1S,2R,3S,4R)-4-(6-[tetrahydro-2H-pyran-4-amino]-2-hydrazino-purin-9-yl)-2,3-dihydroxy-cyclopentyl]-propionamide according to the process as described by Elzein et al at page 163, Scheme 4 Intermediate 6 of Bioorg. Med. Chem. Lett. 2007, 17, 161-166, gives the title compound.

### EXAMPLE 13

### 4-[({1-[6-[(1S,2S)-2-methoxycyclopentylamino]-9-((1R,2S,3R,4S)-2,3-dihydroxy-4-propionylamino-cyclopentyl)-9Hpurin-2-yl]-1H-pyrazole-4-carbonyl}-amino)-methyl]-benzoic acid

### Step 1: 1-[6-[(1S,2S)-2-methoxycyclopentylamino]-9-((1R,2S,3R,4S)-2,3-dihydroxy-4-propionylamino-cyclopentyl)-9H-purin-2-yl]-1H-pyrazole-4-carboxylic acid

The title compound is prepared in an analogous manner to 1-]6-[tetrahydro-2H-pyran-4-amino]-9-((1R,2S,3R,4S)-2,3-dihydroxy-4-propionylamino-cyclopentyl)-9H-purin-2-yl]-1H-pyrazole-4-carboxylic acid (Example 12).

### Step 2: 4-[({1-[6-[(1S,2S)-2-methoxycyclopentylamino]-9-((1R,2S,3R,4S)-2,3-dihydroxy-4-propionylamino-cyclopentyl)-9Hpurin-2-yl]-1H-pyrazole-4-carbonyl}-amino)-methyl]-benzoic acid

Amide bond formation with 1-[6-[(1S,2S)-2-methoxycyclopentylamino]-9-((1R,2S,3R,4S)-2,3-dihydroxy-4-propionylamino-cyclopentyl)-9H-purin-2-yl]-1H-pyrazole-4-carboxylic acid, as described by Elzein et al at page 163, scheme 3 of Bioorg. Med. Chem. Lett. 2007, 17, 161-166, gives the title compound.

### EXAMPLE 14

### 1-[9-[(1R,2S,3R,4S)-2,3-Dihydroxy-4-(2-hydroxy-acetylamino)-cyclopentyl]-6-(3-fluoro-4-hydroxy-phenylamino)-9H-purin-2-yl]-1H-pyrazole-4-carboxylic acid amide

The title compound is prepared in an analogous manner to 1-[6-cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride (Example 5)

### EXAMPLE 15

### 1-{6-[(1S,2S)-2-methoxycyclopentylamino]-9-[(1R,2S,3R,4S)-2,3-dihydroxy-4-(5-methyl-tetrazol-2-yl)cyclopentyl]9Hpurin-2-yl}-1H-pyrazole-4-carboxylic acid amide

### Step 1: (1R,2S,3R,5S)-3-(6-[(1S,2S)-2-methoxycyclopentylamino]-2-chloro-purin-9-yl)-5-(5-methyl-tetrazol-2-yl)-cyclopentane-1,2-diol

The title compound is prepared in an analogous manner to (1R,2S,3R,5S)-3-[2-chloro-6-[(1S,2S)-2-methoxycyclopentylamino]-purin-9-yl]-5-(5-ethyl-tetrazol-2-yl)-cyclopentane-1,2-diol (Example 10) using 5-methyl-2H-tetrazole in place of 5-ethyl-2H-tetrazole.

### Step 2: 1-{6-([1S,2S)-2-methoxycyclopentylamino]-9-[(1R,2S,3R,4S)-2,3-dihydroxy-4-(5-methyl-tetrazol-2-yl)cyclopentyl]-9H-purin-2-yl}-1H-pyrazole-4-carboxylic acid amide

Introduction of the pyrazole carboxamide into (1R,2S,3R,5S)-3-(6-[(1S,2S)-2-methoxycyclopentylamino]-2-chloro-purin-9-yl)-5-(5-methyl-tetrazol-2-yl)-cyclopentane-1,2-diol is carried out in an analogous manner to the preparation of 1-[6-cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride (Example 5).

### EXAMPLE 16 (EX-P11)

### N-[(1S,2R,3S,4R)-4-(6-(1R,2S,4S)-bicyclo of [2.2.1]heptan-2-amino-2-pyrazol-1-yl-purin-9-yl)-2,3-dihydroxy-cyclopentyl]-2-hydroxy-acetamide

The title compound is prepared in an analogous manner to 1-[6-cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride (Example 5).

### EXAMPLE 17

### (1R,2S,3R,5S)-3-[6-((1S,2S)-2-Hydroxy-cyclopentylamino)-2-(4-pyridin-2-yl-pyrazol-1-yl-purin-9-yl]-5-(4-hydroxymethyl-pyrazol-1-yl-cyclopentane-1,2-diol

### Step 1: (1R,2S,3R,5S)-3-[2-Hydrazino-6-((1S,2S)-2-hydroxy-cyclopentylamino)-purin-9-yl]-5-(4-hydroxymethyl-pyrazol-1-yl)-cyclopentane-1,2-diol

The title compound is prepared from (1R,2S,3R,5S)-3-[2-chloro-6-[(1S,2S)-2-hydroxycyclopentylamino]-purin-9-yl]-5-(4-hydroxymethyl-pyrazol-1-yl)-cyclopentane-1,2-diol in an analogous manner to N-[(1S,2R,3S,4R)-4-(6-[tetrahydro-2H-pyran-4-amino]-2-hydrazino-purin-9-yl)-2,3-dihydroxy-cyclopentyl]-propionamide (Example 12, step 2).

### Step 2: (1R,2S,3R,5S)-3-[6-(1S,2S)-2-Hydroxy-cyclopentylamino)-2-(4-pyridin-2-yl-pyrazol-1-yl)-purin-9-yl]-5-(4-hydroxymethyl-pyrazol-1-yl)-cyclopentane-1,2-diol

Pyrazole formation with (1R,2S,3R,5S)-3-[2-hydrazino-6-((1S,2S)-2-hydroxy-cyclopentylamino)-purin-9-yl]-5-(4-hydroxymethyl-pyrazol-1-yl)-cyclopentane-1,2-diol and 2-pyridyl substituted malonaldehyde, prepared according to the process described by Elzein et al at page 162, scheme 1 of Bioorg. Med. Chem. Lett. 2007, 17, 161-166, gives the title compound.

### EXAMPLE 18

### 1-{9-[(1R,2S,3R,4S)-2,3-Dihydroxy-4-(2-hydroxy-acetylamino)-cyclopentyl]-6-[(R)-(tetrahydro-furan-3-yl)amino]-9H-purin-2-yl}-1H-pyrazole-4-carboxylic acid methylamide

The title compound is prepared in an analogous manner to 4-[({1-[6-[(1S,2S)-2-methoxycyclopentylamino]-9-((1R,2S,3R,4S)-2,3-dihydroxy-4-propionylamino-cyclopentyl)-9Hpurin-2-yl]-1H-pyrazole-4-carbonyl}-amino)-methyl]-benzoic acid (Example 13) using (3R)-tetrahydro-furanamine in place of cyclopentylamine.

### EXAMPLE 19

### 1-[9-[(1R,2S,3R,4S)-2,3-Dihydroxy-4-(2-hydroxy-acetylamino)-cyclopentyl]-6-((1S,2S)-2-methoxy-cyclopentylamino)-9H-purin-2-yl]-1H-pyrazole-4-carboxylic acid amide

The title compound is prepared in an analogous manner to 1-[6-cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride (Example 5).

### EXAMPLE 20

### N-((1S,2R,3S,4R)-4-{6-[(R)-1-(3-Chloro-thiophen-2-ylmethyl)-propylamino]-purin-9-yl}-2,3-dihydroxy-cyclopentyl)-acetamide

The title compound is prepared in an analogous manner to N-[(1S,2R,3S,4R)-4-(6-cyclopentylamino-purin-9-yl)-2,3-dihydroxy-cyclopentyl]-propionamide (Example 1) using (R)-(3-chloro-2-thienyl)-2-butylamine in place of cyclopentylamine (prepared according to the procedure illustrated in ‘Synthesis of a potent A1 selective adenosine agonist: N6-[1-R-[(3-chloro-2-thienyl)methyl]propyl]adenosine, RG 14718(−)’ Fink et al Nucleosides and Nucleotides 1992, 11, 1077-1088).

### EXAMPLE 21

### (1S,2R,3S,5R)-3-(4-Methyl-[1,2,3]triazol-2-yl)-5-{6-[(S)-1-(5-trifluoromethyl-pyridin-2-yl)-pyrolidin-3-ylamino]-purin-9-yl}-cyclopentane-1,2-diol

### Step 1: (1R,2S,3R,5S)-3-{2-Chloro-6-[1-(5-trifluoromethyl-pyridin-2-yl)-pyrrolidin-3-ylamino]-purin-9-yl}-5-(4-methyl-[1,2,3]triazol-2-yl)-cyclopentane-1,2-diol

The title compound is prepared in an analogous manner to the process illustrated in WO 2006/074925 at page 41, Intermediate BA1 for the preparation of (1R,2S,3R,5S)-3-[2-chloro-6-(2,2-diphenyl-ethylamino)-purin-9-yl]-4-methyl-pyrazol-1-yl)-cyclopentane-1,2-diol, by replacing 4-methylpyrazole with 4-methyl-1,2,3-triazole and 2,2-diphenylethylamine with (35)-1-[5-(trifluoromethyl)-2-pyridinyl]-3-pyrrolidinamine (prepared according to the procedure illustrated in WO 1998/001426, at page 35, Example 3, step 4).

### Step 2: (1S,2R,3S,5R)-3-(4-Methyl-[1,2,3]triazol-2-yl)-5-{6-[(S)-1-(5-trifluoromethyl-pyridin-2-yl)-pyrrolidin-3-ylamino]-purin-9-yl}-cyclopentane-1,2-diol

Hydrogenation of (1R,2S,3R,5S)-3-{2-Chloro-6-[1-(5-trifluoromethyl-pyridin-2-yl)-pyrrolidin-3-ylamino]-purin-9-yl}-5-(4-methyl-[1,2,3]triazol-2-yl)-cyclopentane-1,2-diol in an analogous manner to that used to prepare Intermediates D and E as illustrated hereinafter gives the title compound.

### EXAMPLE 22

### N-((1S,2R,3S,4R)-4-{6-[4-({-4-[(2-Amino-ethylcarbamoyl)-methyl]-phenylcarbamoyl}-methyl)-phenylamino]-purin-9-yl}-2,3-dihydroxy-cyclopentyl)-propionamide

The title compound is prepared from Intermediates B and C following the procedures used to prepare N-[(1S,2R,3S,4R)-4-(2-chloro-6-cyclopentylamino-purin-9-yl)-2,3-dihydroxy-cyclopentyl]-propionamide (Example 1) in combination with those reported by Jacobsen et al (J. Med. Chem. 1988, 28, 1341-1346.).

### EXAMPLES 23-26

These compounds namely,
N-[(1S,2R,3S,4R)-4-(6-((1R,2S,4S)-bicyclo[2.2.1]heptan-2-amino)-purin-9-yl)-2,3-dihydroxy-cyclo pentyl]-propionamide (Ex. 23),
N-[(1S,2R,3S,4R)-4-(6-Cyclohexylamino-purin-9-yl)-2,3-dihydroxy-cyclopentyl]-propionamide (Ex. 24),
N-((1S,2R,3S,4R)-4-{6-[(R)-1-(4-Chloro-thiophen-3-ylmethyl)-propylamino]purin-9-yl}-2,3-dihydroxy-cyclopentyl)-propionamide (Ex. 25) and
N-((1S,2R,3S,4R)-2,3-Dihydroxy-4-{6-[(R)-(tetrahydro-furan-3-yl)amino]-purin-9-yl}-cyclopentyl)-propionamide (Ex. 26)
   are prepared from Intermediates B and C in an analogous sequence to that used to prepare Example 1 by replacing cyclopentylamine with the following amines: (Ex 23) (1R,2S,4S)-bicyclo[2.2.1]heptan-2-amine (prepared according to the method illustrated at page 10, preparation VI, of EP 2911051); (Ex 24) cyclohexylamine; (Ex 25) (R)-(3-chloro-2-thienyl)-2-butylamine (prepared according to the procedure illustrated in ‘Synthesis of a potent A1 selective adenosine agonist: N6-[1-R-[(3-chloro-2-thienyl)methyl]propyl]adenosine, RG 14718(−)’ Fink et al Nucleosides and Nucleotides 1992, 11, 1077-1088); and (Ex 26) (3R)-tetrahydro-furanamine as necessary.

### EXAMPLE 27

### N-((1S,2R,3S,4R)-4-{6-[(R)-2-(Benzothiazol-2-ylsulfanyl)-1-methyl-ethylamino]-2-chloro-purin-9-yl}-2,3-dihydroxy-cyclopentyl)-propionamide

The title compound is prepared in an analogous manner to the process for the preparation of N-[(1S,2R,3S,4R)-4-(2-chloro-6-cyclopentylamino-purin-9-yl)-2,3-dihydroxy-cyclopentyl]-propionamide (Example 1) as described to prepare compound 12, scheme 2 in Knutsen et al J. Med. Chem. 1999, 42, 3463-3477, by using (S)-1-(2-benzothiazolylthio)-2-propanamine in place of cyclopentylamine.

### EXAMPLE 28

### N-{(1S,2R,3S,4R)-2,3-Dihydroxy-4-[6-((1S,2S)-2-hydroxy-cyclopentylamino)-purin-9-yl]-cyclopentyl}-propionamide

This compound is prepared from Intermediates B and C in an analogous sequence to that used to prepare Example 1 by replacing cyclopentylamine with (1S,2S)-2-amino-cyclopentanol.

### EXAMPLE 29

### Biphenyl-4-carboxylic acid [9-((1R,2S,3R,4S)-2,3-dihydroxy-4-propionylamino-cyclopentyl)-9H-purin-6-yl]-amide

### Step 1: Biphenyl-4-carboxylic acid [2-chloro-9-((1R,2S,3R,4S)-2,3-dihydroxy-4-propionylamino-cyclopentyl)-9H-purin-6-yl]-amide

The title compound is prepared by the acylation of N-[(1S,2R,3S,4R)-4-(6-amino-2-chloro-9H-purin-9-yl)-2,3-dihydroxycyclopentyl]-propanamide (WO 2006/045552) according to the procedure used to prepare compound 9 of Baraldi et al (J. Med. Chem. 1998, 41, 3174-3185.).

### Step 2: Biphenyl-4-carboxylic acid [9-((1R,2S,3R,4S)-2,3-dihydroxy-4-propionylamino-cyclopentyl)-9H-purin-6-yl]-amide

Hydrogenation of biphenyl-4-carboxylic acid [2-chloro-9-((1R,2S,3R,4S)-2,3-dihydroxy-4-propionylamino-cyclopentyl)-9H-purin-6-yl]-amide in an analogous manner to that used to prepare Intermediates D and E gives the title compound.

### EXAMPLE 30

### N-{(1S,2R,3S,4R)-4-[2-Chloro-6-((R)-1-methyl-2-phenoxy-ethylamino)-purin-9-yl]-2,3-dihydroxy-cyclopentyl}-propionamide

The title compound is prepared in an analogous manner to the process for the preparation of N-[(1S,2R,3S,4R)-4-(2-chloro-6-cyclopentylamino-purin-9-yl)-2,3-dihydroxy-cyclopentyl]-propionamide (Example 1) using (R)-1-phenoxy-2-propanamine, as described in Knutsen et al J. Med. Chem. 1999, 42, 3463-3477 to prepare compound II, scheme 1, by using 1-methyl-2-phenoxy-ethylamine in place of cyclopentylamine.

### EXAMPLE 31

### N-{(1S,2R,3S,4R)-4-[2-Chloro-6-(4-phenylsulfanyl-piperidin-1-ylamino)-purin-9-yl]-2,3-dihydroxy-cyclopentyl}-propionamide

The title compound is prepared in an analogous manner to the process for the preparation of N-[(1S,2R,3S,4R)-4-(2-chloro-6-cyclopentylamino-purin-9-yl)-2,3-dihydroxy-cyclopentyl]-propionamide, as described to prepare compound 13, scheme 3 of Knutsen et al J. Med. Chem. 1999, 42, 3463-3477, by using 4-(phenylthio)-1-piperidinamine in place of cyclopentylamine.

### EXAMPLE 32

### N-[(1S,2R,3S,4R)-4-(2-[(1E)-3-[(Phenylamino)carbonyl]-1-triazenyl]-6-cyclopentylamino-purin-9-yl)-2,3-dihydroxy-cyclopentyl-propionamide

The title compound is prepared from Intermediate D following the procedure of Beukers et al (J. Med. Chem. 2003, 46, 1492-1503) in combination with the acylation reaction used to prepare N-[(1S,2R,3S,4R)-4-(6-cyclopentylamino-purin-9-yl)-2,3-dihydroxy-cyclopentyl]-propionamide (Example 1).

### EXAMPLE 33

### 1-[6-((1R,2S,4S)-Bicyclo[2.2.1]hept-2-ylamino)-9-((1R,2S,3R,4S)-2,3-dihydroxy-4-propionylamino-cyclopentyl)-9H-purin-2-yl]-1H-pyrazole-4-carboxylic acid

The title compound is prepared in an analogous manner to 1-[6-{tetrahydro-2H-pyran-4-amino}-9-((1R,2S,3R,4S)-2,3-dihydroxy-4-propionylamino-cyclopentyl)-9H-purin-2-yl]-1H-pyrazole-4-carboxylic acid (as illustrated in EP 2911051, at page 10, preparation VI) by substituting tetrahydro-2H-pyran-4-amine with (1R,2S,4S)-bicyclo[2.2.1]heptan-2-amine.

### EXAMPLE 34

### 1-{9-((1R,2S,3R,4S)-2,3-Dihydroxy-4-propionylamino-cyclopentyl)-6-[(R)-(tetrahydro-furan-3-yl)amino]-9H-purin-2-yl}-1H-pyrazole-4-carboxylic acid amide

The title compound is prepared in an analogous manner to 1-[6-cyclopentylamino-9-((1R,2S,3R,4S)-2,3-dihydroxy-4-propionylamino-cyclopentyl)-9H-purin-2-yl]-1H-pyrazole-4-carboxylic acid amide by substituting cyclopentylamine with (3R)-tetrahydro-furanamine.

### Preparation of Intermediate Compounds

### Intermediate A

### Dibenzyl (1S,2R,3S,4R)-4-(2,6-dichloro-9H-purin-9-yl)-2,3-dihydroxycyclopentyl iminodicarbonate

### A1: Dibenzyl Iminodicarbonate

A cooled (0° C.) solution of benzyl carbamate (4.0 g, 27 mmol) in THF (100 ml) under an inert atmosphere of argon is treated with potassium iodide (3.2 g of a 35% w/w dispersion in oil, 28 mmol) portion-wise over 10 minutes. The reaction mixture is allowed to warm to RT over 30 minutes after which time benzyl chloroformate (5.0 g, 29 mmol) is added. After stirring at RT for 2 hours, the reaction is quenched with water (20 ml). The THF is removed in vacuo and the resulting mixture is partitioned between EtOAc and 2M HCl. The organic portion is separated and washed with brine, dried (MgSO₄) and concentrated in vacuo. The resulting oil is purified by chromatography on silica eluting with 1:3 EtOAc/iso-hexane to yield a product which is recrystallised from DCM/iso-hexane to afford the title product. [M+H]+ 286.

### A2: Dibenzyl (1S,4R)-4-(2,6-dichloro-9H-purin-9-yl)cyclopent-2-enyliminodicarbonate

A solution comprising carbonic acid (1S,4R)-4-(2,6-dichloro-purin-9-yl)-cyclopent-2-enyl ester ethyl ester (as illustrated in WO 2006/045552, at page 54, Example 4, step 2) (2.0 g, 5.83 mmol), dibenzyl iminodicarbonate (A1) (2.2 g, 7.58 mmol) and triphenyl phosphine (229 mg, 0.9 mmol) in THF (20 ml) is stirred at RT for 30 minutes.

Tris(dibenzylideneacetone)dipalladium (0) (238 mg, 0.3 mmol) is added and the resulting mixture is stirred at RT for 1.5 hours. The solvent is removed in vacuo and the crude product is purified by chromatography on silica eluting with MeOH/DCM (gradient of 0 to 1% MeOH) to yield the title compound. [M+H]+ 538.

### A3: Dibenzyl (1S,2R,3S,4R)-4-(2,6-dichloro-9H-purin-9-yl)-2,3-dihydroxycyclopentyl iminodicarbonate

A rapidly stirred, cooled (4° C.) solution of dibenzyl (1S,4R)-4-(2,6-dichloro-9H-purin-9-yl)cyclopent-2-enyliminodicarbonate (A2) (8.96 g) in EtOAc (150 ml), MeCN (150 ml) and water (50 ml) is treated with sodium periodate (5.33 g). The mixture is warmed to 35° C. to aid dissolution of the sodium periodate and then re-cooled to 4° C. Ruthenium trichloride is added in one portion and the reaction mixture is stirred for 10 minutes at 4° C. Sodium bisulphite solution is added (45 g in 90 ml water) and the mixture is stirred rapidly for 1.5 hour and allowed to warm to RT. The mixture is then extracted with EtOAc (2×250 ml) and the organic extracts are washed with water, brine, dried (MgSO₄) and concentrated in vacuo. The resulting foam is dissolved in MeOH and filtered, and the filtrate is concentrated in vacuo. The resulting foam is triturated with iso-hexane (150 ml) followed by ether (150 ml) to afford the title compound. [M+H]+ 572.

### Intermediates B and C

### Dibenzyl (1S,2R,3S,4R)-4-(2-chloro-6-(cyclopentylamino)-9H-purin-9-yl)-2,3-dihydroxycyolopentylcarbamate (Intermediate B) and Benzyl (1S,2R,3S,4R)-4-(2-chloro-6-(cyclopentylamino)-9H-purin-9-yl)-2,3-dihydroxycyolopentylcarbamate (Intermediate C)

A stirred solution of dibenzyl (1S,2R,3S,4R)-4-(2,6-dichloro-9H-purin-9-yl)-2,3-dihydroxycyclopentyl iminodicarbonate (Intermediate A) (572 mg) in THF (1 ml) at RT is treated with cyclopentylamine (247 μl) and stirred overnight. The resulting mixture is partitioned between 0.5M HCl and DCM and the organic layer is separated. The aqueous portion is extracted further with DCM (3×) and the organic extracts are combined, dried (MgSO₄) and concentrated in vacuo. The crude residue comprises a mixture of products which are separated by chromatography on silica eluting with 2% MeOH in DCM afford to afford (Intermediate B) as a light brown glassy solid. The solvent gradient is increased to 4% MeOH in DCM to afford the second product benzyl (1S,2R,3S,4R)-4-(2-chloro-6-(cyclopentylamino)-9H-purin-9-yl)-2,3-dihydroxycyclopentylcarbamate (Intermediate C) as a brown/white amorphous solid.

### Intermediates D and E

### (1S,2R,3S,5R)-3-Amino-5-(6-cyclopentylamino-purin-9-yl)-cyclopentane-1,2-diol (Intermediate D) and (1S,2R,3S,5R)-3-Amino-5-(2-chloro-6-cyclopentylamino-purin-9-yl)-cyclopentane-1,2-diol (Intermediate E)

A stirred solution of a 1:1 mixture of dibenzyl (1S,2R,3S,4R)-4-(2-chloro-6-(cyclopentylamino)-9H-purin-9-yl)-2,3-dihydroxycyclopentylcarbamate (Intermediate B) and benzyl (1S,2R,3S,4R)-4-(2-chloro-6-(cyclopentylamino)-9H-purin-9-yl)-2,3-dihydroxycyclopentylcarbamate (Intermediate C) (370 mg) in EtOH (10 ml) under an inert atmosphere of argon is treated with Palladium (10% on charcoal) (37 mg). The reaction mixture is placed under an atmosphere of hydrogen and stirred at RT. After 6 hours, the mixture is filtered and the residue is washed with 1:1 2M HCl/MeOH. The filtrate is concentrated in vacuo and purification of the crude residue by reverse phase column chromatography (Isolute™ C18, 0-60% acetonitrile in water −0.1% HCl) affords (1S,2R,3S,5R)-3-amino-5-(6-cyclopentylamino-purin-9-yl)-cyclopentane-1,2-diol hydrochloride [M+H]⁺ 319 and (1S,2R,3S,5R)-3-amino-5-(2-chloro-6-cyclopentylamino-purin-9-yl)-cyclopentane-1,2-diol hydrochloride.

### Intermediate F

### 1H-Pyrazole-4-carboxylic acid amide

A mixture of ethyl pyrazole-4-carboxylate (1.00 g) in 880 ammonia (10 ml of a 1.4 mmol/ml solution) is allowed to stand at RT for three days. The resulting suspension is filtered, dissolved in MeOH (10 ml), and allowed to stand at RT until the solvent had reduced in volume to yield the title compound as a white crystalline solid.

### Intermediate G

### Preparation of (1H-pyrazol-4-yl)-methanol

4-Ethylpyrazole carboxylate (10 g, 71.40 mmol) is placed in an oven-dried flask under an atmosphere of argon. Dry THF (100 ml) is added followed by the dropwise addition of lithium aluminium hydride (1 M in THF, 100 ml, 100 mmol). Once the addition is complete the reaction mixture is stirred at 50° C. The reaction is shown to be complete by NMR after 4 hours. The reaction mixture is cooled on an ice-bath and the reaction mixture is quenched with water (3.8 ml) then 15% sodium hydroxide (3.8 ml) and finally water again (11.4 ml). The solvent is removed in vacuo and the solid is placed in a Soxhlet apparatus. THF is refluxed through the system for 24 hours. The solvent is removed in vacuo to give the title compound. ¹H NMR (MeOD, 400 MHz); 7.60 (s, 2H), 4.55 (s, 2H).

### Data

Table 2 illustrates pEC₅₀ data for compounds of the invention. The pEC₅₀ figures listed represent the mean of >2 measurements, wherein the data was obtained according to the methodology described hereinbefore.

TABLE 2

| Example | Mean pEC₅₀ |
|---|---|
| 1 | 4.79 × 10⁻⁸ |
| 2 | 6.77 × 10⁻⁸ |
| 3 | 9.79 × 10⁻⁸ |
| 4 | 4.01 × 10⁻⁸ |

1. A compound of formula (I) 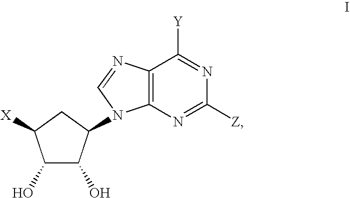
or stereoisomers thereof, in free or pharmaceutically acceptable salt form, wherein
X is —NHC(O)R¹, —NHC(O)OR², N-boned HET¹, or, NHC(O)—NR³R⁴
wherein R¹ and R² are independently selected from the group including H, C₁-C₈ alkyl, C₂-C₈ alkenyl, C₂-C₈ alkynyl, C₁-C₈ alkoxy, and C₃-C₈ cycloalkyl, and wherein said alkyl, alkenyl, alkynyl, alkoxy or cycloalkyl groups of R¹ and R² may optionally be substituted by one or more substituents independently selected from the group including NH₂, OH, and OR⁵, and wherein R⁵ is a C₁-C₃ alkyl group;
wherein R³ and R⁴ are independently selected from the group including H, and C₁-C₄ alkyl; wherein said HET¹ group is an N-bonded 4- to 6-membered heterocyclic group containing from 1 to 4 nitrogen atoms and may optionally be benzo-fused, and wherein HET¹ may optionally be substituted by one or more groups independently selected from the group including H, C₁-C₃ alkyl, C₁-C₃ alkoxy, and —C(O), and wherein said alkyl and alkoxy groups may optionally be further substituted by —NH₂ or —OH;
Y is —NH₂, —NHR⁶, —N(R⁶)₂, —NHR⁶(aryl), —NHR⁷(HET²), —NHR⁸, —NHC(O)R⁸, or —NH(HET³),
wherein R⁶ is C₁-C₈ alkyl, C₂-C₈ alkenyl, C₂-C₈ alkynyl, C₁-C₈ alkoxy, or C₃-C₈ cycloalkyl group, and wherein said cycloalkyl group may be saturated or unsaturated, fused or bridged, and wherein said alkyl, alkenyl, alkynyl, alkoxy, or cycloalkyl groups of R⁶ may be optionally substituted by one or more groups independently selected from the group including OH, halogen, —C₁-C₆alkoxy, —C₁-C₆alkyl, —O-aryl, and an —S—(S-HET) heterocyclic group, and wherein —(S-HET) is a C-bonded 5- to 8-membered ring system having one or two heteroatoms selected from O, N and S, and wherein —(S-HET) may be optionally substituted by one or more groups independently selected from halogen, and C₁-C₈ alkyl;
wherein the HET² group of —NHR⁷(HET²) is a C-bonded 5- or 6-membered heterocyclic group containing one or two heteroatoms selected from O, N and S, and wherein HET² may optionally be substituted by one or more substitutents independently selected from the group including halogen, C₁-C₆alkyl, and —C(O)C₁-C₆alkyl;
wherein R⁷ is a C₁-C₈ alkyl group which may be optionally substituted by a C₁-C₃ alkyl group;
wherein the HET³ group of —NH(HET³) is a C or N-bonded 5- or 6-membered heterocyclic group containing one or two heteroatoms selected from O, N and S, and wherein HET³ may optionally be substituted by one or more substituents independently selected from the group including halogen, —C₁-C₆alkyl, —C(O)O(C₁-C₆alkyl), —S-aryl, and a —C-bonded 5- or 6-membered heterocyclic group containing one or two N heteroatoms (C-HET¹) wherein C-HET¹ is optionally substituted by one or more CF₃ substitutents;
wherein R⁸ is an aryl group
wherein the aryl group of —NHR⁸ is either mono-substituted with —OH, halogen or —C₁-C₆alkyl, or di-substituted with two groups independently selected from the group including —OH, halogen, —C₁-C₆alkyl, —N(—C₁-C₆alkyl)₂, and —NH(HET⁴); or is tri-substituted with three groups independently selected from the group including —OH, halogen, and —C₁-C₆alkyl; wherein said HET⁴ group is a C-bonded 5- or 6-membered heterocyclic group containing one or two heteroatoms selected from O, N and S, and wherein HET⁴ may optionally be substituted by one or more groups independently selected from the group including H, halogen, —C₁-C₆alkyl, aryl, heteroaryl, —C₁-C₆alkoxy, —O-aryl, —N(C₁-C₆alky), —N(aryl), and —N(heteroaryl);
wherein the aryl group of —NHC(O)R⁸ may be optionally substituted by one or more aryl groups;
Z is H, halogen, HET⁵, or —N═N—NHC(O)—NH-aryl, wherein said HET⁵ group is a 5- or 6-membered ring containing from one to four N heteroatoms, and wherein HET⁵ may optionally be substituted by one or more groups independently selected from the group including —C₁-C₆alkyl-C(O)R^{x}, —C(O)R^{x}, —C(O)NHR^{y}, —NHC(O)R^{x}, a C-bonded 5- or 6-membered ring containing from one or two N heteroatoms (HET⁵), and aryl;
wherein R^{x} is selected from the group including H, OH, C₁-C₆alkyl, —O(C₁-C₆alkyl), and aryl,
wherein said aryl group may be optionally substituted by halogen or C₁-C₃alkyl; and
wherein R^{y} is selected from the group including H, C₁-C₆alkyl, aryl, and C₁-C₆alkyl(aryl), wherein said aryl groups may be optionally substituted by one or more CF₃ groups.

2. A compound according to claim 1 wherein
X is NHC(O)R¹, —NHC(O)OR², N-bonded HET¹, or —NHC(O)—NR³R⁴;
wherein R¹ and R² are independently selected from the group including C₁-C₄ alkyl, C₁-C₃ alkoxy, and C₃-C₄ cycloalkyl, and wherein said alkyl, alkoxy or cycloalkyl groups may optionally be substituted by one or more substituents independently selected from NH₂, and OH;
wherein R³ and R⁴ are independently selected from H, and methyl;
wherein HET¹ is an, optionally benzo-fused, N-bonded 5- to 6-membered heterocyclic group containing from 1 to 4 N heteroatoms, and wherein HET¹ may optionally be substituted by one or more groups independently selected from the group including H, methyl, ethyl, i-propyl, n-propyl, —CH₂OH, —OCH₃, —CH₂CH₂OH, —CH₂NH₂, —CH(CH₃)OH, and —C(O); and wherein
Y is —NH₂, —NHR⁶, —N(R⁶)₂, —NHR⁷(HET²), —NHR⁸, —NHC(O)R⁸, or —NH(HET³), wherein R⁶ is C₁-C₄ alkyl, or C₃-C₈ cycloalkyl wherein said cycloalkyl group may be saturated, fused or bridged; and
wherein, when Y is —NHR⁶, R⁶ is selected from the group including Me, Et, iPr, nPr, iBu, nBu, tBu, and C₃-C₈ cycloalkyl, or R⁶ is a C₁ to C₄ alkyl group substituted by —S—(S-HET) or —O-aryl; and
wherein, when Y is —N(R⁶)₂, R⁶ is C₃-C₅ cycloalkyl, and
wherein said alkyl, or cycloalkyl groups of NHR⁶ and N(R⁶)₂ may be optionally substituted by one or more groups independently selected from the group including halogen, —C₁-C₃alkoxy, —C₁-C₃alkyl, —O-aryl, and —S—(S-HET), and
wherein, when Y is —NHR⁷(HET²), R⁷ is C₁-C₄ alkyl and HET² is a C-bonded 5-membered heterocyclic group containing one heteroatom selected from O, S and N, and wherein HET² may optionally be substituted by one or more substitutents independently selected from the group including Cl, F, Me, and Et, and wherein the alkyl group of —NHR⁷(HET²) is optionally substituted by a C₁-C₃ alkyl group; and
wherein the 5- to 6-membered heterocyclic group of —NH(HET³) is C- or N-bonded and contains one or two heteroatoms selected from O, and N, and, may optionally be substituted by one or more substituents independently selected from the group including Cl, F, —C₁-C₃alkyl, —C(O)O(C₁-C₃alkyl), —S-phenyl, and —C-HET¹ wherein —C-HET¹ is a C-bonded 6-membered heterocyclic group containing one N heteroatom and wherein —C-HET¹ is optionally substituted by one or more —CF₃ substitutents,
wherein R⁸ is a phenyl group; and
wherein the phenyl group of —NHR⁸ is either: mono-substituted with —OH, F, Cl, —C₁-C₃alkyl, or —CH₂C(O)NH-phenyl-C(O)NH—CH₂NH₂; or is di-substituted with two groups independently selected from the group including —OH, F, Cl, and —C₁-C₃alkyl; or is tri-substituted with three groups independently selected from the group including —CH₃, F, and, Cl, and
wherein the phenyl group of —NHC(O)R⁸ may be optionally substituted by one or more aryl groups;
and wherein
Z is H, Cl, F or HET⁵ wherein HET⁵ is an N-bonded 5-membered heterocyclic group containing one or two N heteroatoms, and wherein HET⁵ is optionally substituted by one or more groups independently selected from —C(O)R^{x}, —C(O)NHR^{y} and a —C-bonded 6-membered heterocyclic group containing one or two N heteroatoms (HET⁶); and
wherein R^{x} is —OMe, —OEt, OH, or phenyl, and
wherein R^{y} is H, Me, Et, phenyl substituted by CF₃, or C₁-C₃ alkylphenyl substituted by CO₂H, Me or CF₃.

3. A compound according to claim 1 or 2, wherein
X is —NHC(O)R¹, or an N-bonded HET¹ group,
wherein R¹ is selected from the group including Me, Et, -EtOH, and -MeOH, and
wherein HET¹ is an N-bonded tetrazolyl, pyrazolyl, triazolyl, indazolyl (benzopyrazolyl), 2,4-di-keto-imidazolyl, or 2-keto-pyridinyl group, and
wherein said R¹ or HET¹ groups may be mono-substituted by a substitutent independently selected from the group including OH, Me, Et, MeOH, and EtOH; and wherein
Y is —NHR⁶, —NHR⁷(HET²), —NHR⁸, —NHC(O)R⁸, or —NH(HET³),
wherein R⁶ is ethyl, n-propyl, i-propyl, n-butyl, 1-butyl, t-butyl, cyclopentyl, cyclohexyl, or norbornane (bicyclic[2.2.1]heptane),
wherein said R⁶ alkyl groups of —NHR⁶ may independently be optionally substituted by one or more groups independently selected from the group including C₁-C₃ alkyl, —S—(S-HET), —O-phenyl, and NH(C₅-C₇)cycloalkyl,
wherein said R⁶ cycloalkyl groups of —NHR⁶ may independently be optionally substituted by one or more groups independently selected from the group including —OH, —OCH₃, —O-aryl, and —S-benzothiazole (benzthiazole); and
wherein HET² is thiophene, optionally substituted by one or more substitutents independently selected from the group including Cl, and F, and wherein
wherein HET³ is tetrahydropyran, tetrahydrofuran or pyrrolidine, each of which may be optionally substituted by one or more substituents independently selected from the group including Cl, F, and a pyridinyl group, wherein said pyridinyl group is optionally substituted by one or more substitutents independently selected from the group including CF₃, Cl and F, and
wherein R⁸ is a phenyl group
wherein the phenyl group of —NHR⁸ is either mono-substituted with —OH, F, Cl, or —C₁-C₃alkyl, or is di-substituted with two groups independently selected from the group including —OH, F, and Cl; and
wherein the phenyl group of —NHC(O)R⁸ may be optionally substituted by a phenyl group; and
wherein
Z is H, Cl or a 1H-pyrazole group (HET⁵), wherein said HET⁵ group may be optionally substituted by —C(O)NHR_{y}, or HET⁵, wherein R^{y} is H, Me or —CH₂-phenyl-CO₂H, and wherein HET⁵ is a C-bonded pyridine-2-yl group.

4. A compound of formula I, according to any of claims 1 to 3 wherein X is selected from the group including propionamide, 2-hydroxy-acetamide, 5-ethyltetrazole, 4-hydrozymethylpyrazole, acetamide, and 4-methyl-[1,2,3]triazole.

5. A compound of formula I, according to any of claims 1 to 4 wherein Y is selected from the group including cyclopentylamino, tetrahydropyran-4-yamino, (S)-2-methoxy-cyclopentylamino, 3-fluoro-4-hydroxy-phenylamino, (S)-norbornaneamino [(S)-(bicyclo[2.2.1]heptaneamino)], (1S,2S)-2-methoxycyclopentylamino, (1S,2S) hydroxycyclopentylamino, tetrahydro-2H-pyran-4-amino, 3-fluoro-4-hydroxy-phenylamino, (R)-(tetrahydro-furan-3-yl)amino, (R)-1-(3-chloro-thiophen-2-ylmethyl)-propylamino, (5)-1-(5-trifluoromethyl-pyridin-2-yl)-pyrrolidin-3-yl-amino, 4-({4-[(2-amino-ethylcarbamoyl)-methyl]-phenylcarbamoyl}-methyl)-phenylamino, cyclohexylamino, (R)-1-(4-chloro-thiophene-3-yl)amino, (R)-2-(benzothiazole-2-ylsulfanyl)-1-methyl-ethylamino, biphenyl-4-carboxylicacid-amino, (R)-1-methyl-2-phenoxy-ethylamino, and 4-phenylsulfonyl-piperidin-1-ylamino.

6. A compound of formula I, according to any of claims 1 to 5 wherein Z is selected from the group including H, Cl, 1H-pyrazole-4-carboxylic acid amide, 1H-pyrazole-4-carboxylic acid, (1H-pyrazole-4-carbonyl-amino)-methyl-benzoic acid, pyrazol-1-yl, 4-pyridin-2-yl-pyrazol-1-yl, 1H-pyrazole-4-carboxylic acid methyl amide, and [(phenylamino)carboyl]-1-trizenyl.

7. A compound of formula IA 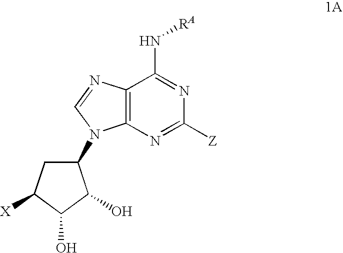
or stereoisomers thereof, in free or pharmaceutically acceptable salt form wherein X and Z are as defined hereinbefore and wherein Y is NH(R^{A}) wherein R^{A} is R⁶, R⁶(aryl), R⁷(HET²), or HET³ wherein R⁶, R⁷HET² and HET³ are each as defined in any of claims 1 to 3.

8. A compound of formula I or IA, according to any of claims 1 to 7 wherein X is selected from the group including propionamide, 2-hydroxy-acetamide, 5-ethyltetrazole, 4-hydrozymethylpyrazole, acetamide, and 4-mathyl-[1,2,3]triazole; and wherein
Y is selected from the group including cyclopentylamino, tetrahydropyran-4-yamino, (S)-2-methoxy-cyclopentylamino, 3-fluoro-4-hydroxy-phenylamino, (S)-norbornaneamino [(S)-(bicyclo[2.2.1]heptaneamino)], (1S,2S)-2-methoxycyclopentylamino, (1S,2S) hydroxycyclopentylamino, tetrahydro-2H-pyran-4-amino, 3-fluoro-4-hydroxy-phenylamino, (R)-(tetrahydro-furan-3-yl)amino, (R)-1-(3-chloro-thiophen-2-ylmethyl)-propylamino, (S)-1-(5-trifluoromethyl-pyridin-2-yl)-pyrrolidin-3-yl-amino, 4-({4-[(2-amino-ethylcarbamoyl)-methyl]-phenylcarbamoyl}-methyl)-phenylamino, cyclohexylamino, (R)-1-(4-chloro-thiophene-3-yl)amino, (R)-2-(benzothiazole-2-ylsulfanyl)-1-methyl-ethylamino, biphenyl-4-carboxylicacid-amino, (R)-1-methyl-2-phenoxy-ethylamino, and 4-phenylsulfonyl-piperidin-1-ylamino; and wherein
Z is selected from the group including H, Cl, 1H-pyrazole-4-carboxylic acid amide, 1H-pyrazole-4-carboxylic acid, (1H-pyrazole-4-carbonyl-amino)-methyl-benzoic acid, pyrazol-1-yl, 4-pyridin-2-yl-pyrazol-1-yl, 1H-pyrazole-4-carboxylic acid methyl amide, and [(phenylamino)carboyl]-1-trizenyl.

9. A compound of formula I, independently selected from:
N-[(1S,2R,3S,4R)-4-(6-Cyclopentylamino-purin-9-yl)-2,3-dihydroxy-cyclopentyl]-propionamide;
N-[(1S,2R,3S,4R)-4-(2-Chloro-6-cyclopentylamino-purin-9-yl)-2,3-dihydroxy-cyclopentyl]-propionamide;
N-[(1S,2R,3S,4R)-4-(6-Cyclopentylamino-purin-9-yl)-2,3-dihydroxy-cyclopentyl]-2-hydroxy-acetamide;
N-[(1S,2R,3S,4R)-4-(2-Chloro-6-cyclopentylamino-purin-9-yl)-2,3-dihydroxy-cyclopentyl]-2-hydroxy-acetamide;
1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride;
N-{(1S,2R,3S,4R)-2,3-Dihydroxy-4-[6-(tetrahydro-pyran-4-ylamino)-purin-9-yl]-cyclopentyl}-propionamide;
N-{(1S,2R,3S,4R)-2,3-Dihydroxy-4-[6-((5)-2-methoxy-cyclopentylamino)-purin-9-yl]-cyclopentyl}-propionamide;
N-{(1S,2R,3S,4R)-4-[6-(3-Fluoro-4-hydroxy-phenylamino)-purin-9-yl]-2,3-dihydroxy-cyclopentyl}-propionamide;
N-{(1S,2R,3S,4R)-2,3-Dihydroxy-4-[6-((1R,2S,4S)-bicyclo[2.2.1]heptan-2-amino)-purin-9-yl]-cyclopentyl}-propionamide,
(1R,2S,3R,5S)-3-[2-Chloro-6-[(1S,2S)-2-methoxycyclopentylamino]-purin-9-yl]-5-(5-ethyl-tetrazol-2-yl)-cyclopentane-1,2-diol;
(1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol;
1-[6-{tetrahydro-2H-pyran-4-amino}-9-((1R,2S,3R,4S)-2,3-dihydroxy-4-propionylamino-cyclopentyl)-9H-purin-2-yl]-1H-pyrazole-4-carboxylic acid tetrahydro-2H-pyran-4-amine
4-[(({1-[6-[(1S,2S)-2-methoxycyclopentylamino]-9-((1R,2S,3R,4S)-2,3-dihydroxy-4-propionylamino-cyclopentyl)-9Hpurin-2-yl]-1H-pyrazole-4-carbonyl}-amino)-methyl]-benzoic acid;
1-[9-[(1R,2S,3R,4S)-2,3-Dihydroxy-4-(2-hydroxy-acetylamino)-cyclopentyl]-6-(3fluoro-4-hydroxy-phenylamino)-9H-purin-2-yl]-1H-pyrazole-4-carboxylic acid amide;
1-{6-[(1S,2S)-2-methoxycyclopentylamino]-9-[(1R,2S,3R,4S)-2,3-dihydroxy-4-(5-methyl-tetrazol-2-yl)cyclopentyl]9Hpurin-2-yl}-1H-pyrazole-4-carboxylic acid amide;
N-[(1S,2R,3S,4R)-4-(6-(1R,2S,4S)-bicyclo[2.2.1]heptan-2-amino-2-pyrazol-1-yl-purin-9-yl)-2,3-dihydroxy-cyclopentyl]-2-hydroxy-acetamide;
(1R,2S,3R,5S)-3-[6-((1S,2S)-2-Hydroxy-cyclopentylamino)-2-(4-pyridin-2-yl-pyrazol-1-yl)-purin-9-yl]-5-(4-hydroxymethyl-pyrazol-1-yl)-cyclopentane-1,2-diol;
1-{9-[(1R,2S,3R,4S)-2,3-Dihydroxy-4-(2-hydroxy-acetylamino)-cyclopentyl]-6-[(R)-(tetrahydro-furan-3-yl)amino]-9H-purin-2-yl}-1H-pyrazole-4-carboxylic acid methylamide;
1-[9-[(1R,2S,3R,4S)-2,3-Dihydroxy-4-(2-hydroxy-acetylamino)-cyclopentyl]-6-((1S,2S)-2-methoxy-cyclopentylamino)-9H-purin-2-yl]-1H-pyrazole-4-carboxylic acid amide;
N-((1S,2R,3S,4R)-4-{6-[(R)-1-(3-Chloro-thiophen-2-ylmethyl)-propylamino]-purin-9-yl}-2,3-dihydroxy-cyclopentyl)-acetamide;
(1S,2R,3S,5R)-3-(4-Methyl-[1,2,3]triazol-2-yl)-5-{6-[(S)-1-(5-trifluoromethyl-pyridin-2-yl)-pyrrolidin-3-ylamino]-purin-9-yl}-cyclopentane-1,2-diol;
N-((1S,2R,3S,4R)-4-[6-[4-({4-[(2-Amino-ethylcarbamoyl)-methyl]-phenylcarbamoyl]-methyl)-phenylamino]-purin-9-yl}-2,3-dihydroxy-cyclopentyl)-propionamide;
N-[(1S,2R,3S,4R)-4-(6-((1R,2S,4S)-bicyclo[2.2.1]heptan-2-amino)-purin-9-yl)-2,3-dihydroxy-cyclo pentyl]-propionamide;
N-[(1S,2R,3S,4R)-4-(6-Cyclohexylamino-purin-9-yl)-2,3-dihydroxy-cyclopentyl]-propionamide;
N-((1S,2R,3S,4R)-4-{6-[(R)-1-(4-Chloro-thiophen-3-ylmethyl)-propylamino]-purin-9-yl}-2,3-dihydroxy-cyclopentyl)-propionamide;
N-((1S,2R,3S,4R)-2,3-Dihydroxy-4-{6-[(R)-(tetrahydro-furan-3-yl)amino]-purin-9-yl}-cyclopentyl)-propionamide;
N-((1S,2R,3S,4R)-4-{6-[(R)-2-(Benzothiazol-2-ylsulfanyl)-1-methyl-ethylamino]-2-chloro-purin-9-yl}-2,3-dihydroxy-cyclopentyl)-propionamide;
N-{(1S,2R,3S,4R)-2,3-Dihydroxy-4-[6-((1S,2S)-2-hydroxy-cyclopentylamino)-purin-9-yl]-cyclopentyl}-propionamide;
Biphenyl-4-carboxylic acid [9-((1R,2S,3R,4S)-2,3-dihydroxy-4-propionylamino-cyclopentyl)-9H-purin-6-yl]-amide;
N-{(1S,2R,3S,4R)-4-[2-Chloro-6-((R)-1-methyl-2-phenoxy-ethylamino)-purin-9-yl]-2,3-dihydroxy-cyclopentyl}-propionamide;
N-{(1S,2R,3S,4R)-4-[2-Chloro-6-(4-phenylsulfanyl-piperidin-1-ylamino)-purin-9-yl]-2,3-dihydroxy-cyclopentyl}-propionamide;
N-[(1S,2R,3S,4R)-4-(2-[(1E)-3-[(Phenylamino)carbonyl]-1-triazenyl]-6-cyclopentylamino-purin-9-yl)-2,3-dihydroxy-cyclopentyl]-propionamide;
1-[6-((1R,2S,4S)-Bicyclo[2.2.1]hept-2-ylamino)-9-((1R,2S,3R,4S)-2,3-dihydroxy-4-propionylamino-cyclopentyl)-9H-purin-2-yl]-1H-pyrazole-4-carboxylic acid;
1-{9-((1R,2S,3R,4S)-2,3-Dihydroxy-4-propionylamino-cyclopentyl)-6-[(R)-(tetrahydro-furan-3-yl)amino]-9H-purin-2-yl}-1H-pyrazole-4-carboxylic acid amide;
and pharmaceutically acceptable salts thereof.

10. A compound of formula I, according to claim 1 independently selected from:
N-[(1S,2R,3S,4R)-4-(6-Cyclopentylamino-purin-9-yl)-2,3-dihydroxy-cyclopentyl]-propionamide;
N-[(1S,2R,3S,4R)-4-(2-Chloro-6-cyclopentylamino-purin-9-yl)-2,3-dihydroxy-cyclopentyl]-propionamide;
N-[(1S,2R,3S,4R)-4-(6-Cyclopentylamino-purin-9-yl)-2,3-dihydroxy-cyclopentyl]-2-hydroxy-acetamide;
N-[(1S,2R,3S,4R)-4-(2-Chloro-6-cyclopentylamino-purin-9-yl)-2,3-dihydroxy-cyclopentyl]-2-hydroxy-acetamide;
1-[6-Cyclopentylamino-9-((1R,2S,3R,4S)-2,3-dihydroxy-4-propionylamino-cyclopentyl)-9H-purin-2-yl]-1H-pyrazole-4-carboxylic acid amide hydrochloride;
N-{(1S,2R,3S,4R)-2,3-Dihydroxy-4-[6-((S)-2-methoxy-cyclopentylamino)-purin-9-yl]-cyclopentyl}-propionamide;
N-{(1S,2R,3S,4R)-2,3-Dihydroxy-4-[6-((1R,2S,4S)-bicyclo[2.2.1]heptan-2-amino)-purin-9-yl]-cyclopentyl}-propionamide
(1R,2S,3R,5S)-3-[6-[(1S,2S)-2-hydroxycyclopentylamino]-purin-9-yl]-5-(4-hydroxymethyl-pyrazol-1-yl)-cyclopentane-1,2-diol;
1-[9-[(1R,2S,3R,4S)-2,3-Dihydroxy-4-(2-hydroxy-acetylamino)-cyclopentyl]-6-(3-fluoro-4-hydroxy-phenylamino)-9H-purin-2-yl]-1H-pyrazole-4-carboxylic acid amide;
N-((1S,2R,3S,4R)-4-{6-[(R)-1-(3-Chloro-thiophen-2-ylmethyl)-propylamino]-purin-9-yl}-2,3-dihydroxy-cyclopentyl)-acetamide;
N-((1S,2R,3S,4R)-4-{6-[(R)-1-(4-Chloro-thiophen-3-ylmethyl)-propylamino]-purin-9-yl}-2,3-dihydroxy-cyclopentyl)-propionamide;
N-((1S,2R,3S,4R)-2,3-Dihydroxy-4-{6-[(R)-(tetrahydro-furan-3-yl)amino]-purin-9-yl}-cyclopentyl)-propionamide;
N-{(1S,2R,3S,4R)-2,3-Dihydroxy-4-[6-((1S,2S)-2-hydroxy-cyclopentylamino)-purin-9-yl]-cyclopentyl}-propionamide;
1-[6-((1R,2S,4S)-Bicyclo[2.2.1]hept-2-ylamino)-9-((1R,2S,3R,4S)-2,3-dihydroxy-4-propionylamino-cyclopentyl)-9H-purin-2-yl]-1H-pyrazole-4-carboxylic acid;
1-{9-((1R,2S,3R,4S)-2,3-Dihydroxy-4-propionylamino-cyclopentyl)-6-[(R)-(tetrahydro-furan-3-yl)amino]-9H-purin-2-yl}-1H-pyrazole-4-carboxylic acid amide;
and pharmaceutically acceptable salts thereof.

11. A compound according to any one of claims 1-10 for use as a pharmaceutical.

12. A compound according to any one of claims 1-10, for use in the treatment of a condition mediated by activation of the adenosine A₁ receptor.

13. A compound according to claim 13, wherein said condition mediated by activation of the adenosine A₁ receptor is type-2 diabetes.

14. A Pharmaceutical composition comprising a compound according to any one of claims 1-10.
